(19) United States
(12) Patent Application Publication
(10) Pub. No.: US 2009/0306625 A1
(43) Pub. Date: Dec. 10, 2009

(54) TREATMENT SYSTEMS AND METHODS FOR RENAL-RELATED DISEASES

(75) Inventors: Nikhil L. Pereira-Kamath, Newton, MA (US); Harry B. Goodson, IV, Fremont, CA (US); Jeffrey M. Elkins, Woodside, CA (US); Samir R. Patel, Mountain View, CA (US)

Correspondence Address: ANGIODYNAMICS, INC., 603 QUEENSBURY AVENUE, QUEENSBURY, NY, 12804, US

(73) Assignee: AngioDynamics, Inc., Queensbury, NY (US)

(21) Appl. No.: 12/404,137

(22) Filed: Mar. 13, 2009

Related U.S. Application Data
(63) Continuation-in-part of application No. 12/044,373, filed on Mar. 7, 2008.
(60) Provisional application No. 60/894,075, filed on Mar. 9, 2007.
(60) Provisional application No. 61/036,350, filed on Mar. 13, 2008.

(51) Int. Cl. A61M 25/01

(52) U.S. Cl. 604/509; 604/522

(57) ABSTRACT

Systems and methods are provided for locally delivering specific prophylactic, regenerative, or therapeutic agents within the body of a patient. Systems and methods can involve direct delivery of prophylactic, regenerative, or therapeutic agents into branch blood vessels or body lumens from a main vessel or lumen, respectively, and in particular into renal arteries extending from an aorta in a patient. Drug infusion techniques encompass specific treatment and prevention regimes for renal diseases including, but not limited to, Acute Kidney Injury, Renal Cell Carcinoma, Polycystic Kidney Disease, and Chronic Kidney Disease.

### CROSS-REFERENCES TO RELATED APPLICATIONS

This application is a continuation-in-part of U.S. patent application Ser. No. 12/044,373, filed Mar. 7, 2008, which claims a benefit of priority to U.S. Patent Application No. 60/894,075, filed Mar. 9, 2007. This application is also a non-provisional of, and claims the benefit of priority to, U.S. Patent Application No. 611036,350, filed Mar. 13, 2008. The entire content of each of the above-referenced filings is incorporated herein by reference.

### BACKGROUND OF THE INVENTION

Embodiments of the present invention relate to the field of medical devices, and more particularly to systems and methods for locally delivering specific prophylactic, regenerative, or therapeutic agents within the body of a patient. Still more particularly, embodiments may relate to systems and methods for locally delivering prophylactic, regenerative, or therapeutic agents into branch blood vessels or body lumens from a main vessel or lumen, respectively, and in particular into renal arteries extending from an aorta in a patient. Drug infusion techniques encompass specific treatment and prevention regimes for renal diseases including, but not limited to, Acute Kidney Injury, Renal Cell Carcinoma, Polycystic Kidney Disease, and Chronic Kidney Disease.

Kidney diseases that can be treated with drug infusions can be broadly divided into, but not limited to, congenital diseases such as polycystic kidney disease (PKD) and congenital urinary tract obstruction; and acquired diseases such as chronic kidney disease (CKD), kidney stones, renal cell carcinoma (RCC), and acute kidney injury (AKI). More particularly, AKI can be further divided into acute tubular necrosis (ATN), acute interstitial nephritis (AIN), and acute glomerulonephritis (AGN).

Acute interstitial nephritis can be an allergic reaction, and can be caused by various pharmaceutical agents. Acute glomerulonephritis can be associated with inflammation and glomerular membrane damage. Acute tubular necrosis typically accounts for 50% of acute renal failure, and can be caused by factors such as prolonged renal hypoperfusion, or nephrotoxic agents.

The prevalence of AKI is approximately 700 k patients yearly with ATN accounting for approximately 560 k patients, AIN for approximately 105 k patients, and GN for approximately 35 k patients. Perhaps more importantly, though, is the prevalence of patients with renal-related diseases who reach end stage renal disease (ESRD) and thus require dialysis or transplantation. Approximately 50,000 GN patients, 17,000 AIN patients, 10,000 PKD patients, 8,000 Tumor patients, and 6,000 TN patients reach ESRD each year. These patients contribute considerably to the $66,650 dialysis, the $99,000 transplant, and $76,500 graft cost per patient in 2004. Causes of acute renal failure can be prerenal, intrinsic, and postrenal.

Many renal diseases involve a cascade of events which cause the up-regulation and down-regulation of proteins and enzymes in the kidney that simultaneously injure the nephrons. Comprehensive therapy can involve drug cocktails to address various routes of damage. Renal diseases are typically treated with either oral administration or intravenous infusion of prophylactic and therapeutic drugs in order to decrease the risk of the injury to the kidney. However, in order to get the desired therapeutic or prophylactic results in the kidney, increased systemic doses are frequently required. A precarious consequence of the increased doses and drug cocktails is the increased vulnerability of the body to the various systemic side effects that accompany these drugs. In addition, many of the systemic side effects can cause further renal damage as a result extrarenal factors that both contribute and lead to nephrotoxicity, renal ischemia, renal inflammation, and various other renal complications. For example, many drugs currently on the market cause hypotension as a common adverse side effect. Systemic hypotension is a leading cause of congestive heart failure, which itself is a particularly common cause of ischemic ATN. Thus, in addition to causing ischemia in the other peripheral organs of the body, and congestive heart failure, most prophylactic drugs administered intravenously are further exacerbating the kidney damage.

Examples of various drugs that have caused systemic and renal complications during prophylactic treatment of the AKI include, but isn't limited to, vasodilators, such as dopamine hydrochloride, fenoldopam mesylate, calcium channel blockers, atrial natriuretic peptides, acetylcholine, nifedipine, nitroglycerine, nitroprusside, adenosine, theophylline; antioxidants such as acetylcysteine, ascorbic acid, glutathione; and diuretics such as mannitol, furosemide; and anti-inflammatory drugs such as NSAIDS, corticosteroids, azathioprine succinate, and cyclophosphamide. Likewise, Renal Cell Carcinoma drugs such as Interleukin-2, Interferon Alpha, Temsirolimus, and Bevacizumab have also shown various unsafe systemic effects. Regrettably, it is apparent that systemic administration of most drugs may be prophylactic or therapeutic for conditions of the kidney is in fact a double edged sword. As a result, the upper dosing of many drugs with therapeutic renal effects is limited by their adverse systemic effects.

Currently, an established intravenous therapy for AKI involves the administration of saline drip (or sodium chloride). Sodium chloride's effects, both prophylactic and adverse, are quite minimal. While no critical adverse effects accompany the systemic infusion of saline, since it is purely a hydration solution, it is neither a treatment nor a prophylaxant for kidney injury. Saline is only considered an established therapy because it “can't hurt”. Working on this hypothesis, companies have manufactured saline drip catheters which fundamentally replace the water lost in urine. Once again, though, there is no significantly evident prophylactic or therapeutic effect of this device.

As is apparent, there is a pressing need for techniques that avoid systemic and renal adverse effects. Saline injection is often not enough. In addition, new modes of administration are needed for stem cells, regenerative drugs, and enzyme regulators. Embodiments of the present invention address at least some of these needs.

### BRIEF SUMMARY OF THE INVENTION

Embodiments of the present invention provide techniques for reliably and accurately localizing agents in the kidney. For example, embodiments provide a targeted therapy system to infuse localized prophylactic and therapeutic drugs directly to the kidneys. One advantage of using local delivery, as opposed to systemic infusion, of prophylactic drugs or reactive agents is that it allows medical professionals to administer a concentrated dosage, either high or low depending on its effects, to the desired region without causing detrimental adverse effects. This targeted therapy can localize the effects of the drug to the region of choice. In addition to the various side effects when administered systemically, systemic administration usually dilutes the impact of the drugs or agents on the desired region. In order to increase the impact, a considerably higher dose is usually required; thus further increasing the risk of injury to the body due to the adverse side effects. Use of a tubular delivery catheter can provide efficient and therapeutic treatment the kidney, especially since current systemic treatments for the kidney have not provided safe nor efficient results.

In one aspect, embodiments of the present invention encompass methods for the treatment of a renal cell carcinoma in a patient in need thereof. Methods may include placing a self-cannulating bifurcated catheter within the abdominal aorta of the patient, wherein the self-cannulating bifurcated catheter includes a first branch extension having a first port and a second branch extension having a second port. Methods may include positioning the self-cannulating bifurcated catheter at or near a first renal artery and a second renal artery of the patient, and allowing the first branch extension of the self-cannulating bifurcated catheter to expand toward the first renal artery, such that the first port of the first branch extension enters the first renal artery. Methods may include allowing the second branch extension of the self-cannulating bifurcated catheter to expand toward the second renal artery, such that the second port of the second branch extension enters the second renal artery. Methods may include infusing a fluid agent through the first and second ports of the first and second branch extensions, respectively, and into the first and second renal arteries of the patient, respectively. The fluid agent can include a therapeutically effective amount of a cytokine. The cytokine can be interleukin-2, or interferon-a. The fluid agent can include both interleukin-2 and interferon-a. Methods may include surgically removing a renal cell carcinoma cell from the patient, and the fluid agent can be administered to the patient following the surgical removal step. In some cases, the patient has been diagnosed with metastatic renal cell carcinoma. In some cases, the fluid agent can include a therapeutically effective amount of an mTOR inhibitor. In some cases, the mTOR inhibitor is Temsirolimus. In some cases, the fluid agent includes a therapeutically effective amount of a mitotic kinesin inhibitor. The mitotic kinesin inhibitor can be Ispinesib. In some cases, the fluid agent includes a therapeutically effective amount of a recombinant humanized antibody to vascular endothelial growth factor. In some cases, the recombinant humanized antibody to vascular endothelial growth factor is Bevacizumab. In some cases, the fluid agent includes a therapeutically effective amount of an anti-transforming growth factor f3 monoclonal antibody. In some cases, the fluid agent includes a therapeutically effective amount of a platelet derived growth factor receptor kinase inhibitor monoclonal antibody. In some cases, the fluid agent comprises a therapeutically effective amount of a stem cell preparation. The stem cell can be a mesenchymal stem cell. Optionally, the stem cell can be an embryonic stem cell. In some cases, the stem cell is an adult stem cell.

In another aspect, embodiments of the present invention encompass the use of interleukin-2 for the manufacture of a medicament for the treatment of renal cell carcinoma in a patient, wherein the treatment includes the administration of the medicament to at least one renal artery of the patient.

In another aspect, embodiments of the present invention encompass the use of interferon-α for the manufacture of a medicament for the treatment of renal cell carcinoma in a patient, wherein the treatment includes the administration of the medicament to at least one renal artery of the patient.

In another aspect, embodiments of the present invention encompass the use of Temsirolimus for the manufacture of a medicament for the treatment of renal cell carcinoma in a patient, wherein the treatment includes the administration of the medicament to at least one renal artery of the patient.

In another aspect, embodiments of the present invention encompass the use of Ispinesib for the manufacture of a medicament for the treatment of renal cell carcinoma in a patient, wherein the treatment includes the administration of the medicament to at least one renal artery of the patient.

In another aspect, embodiments of the present invention encompass the use of Bevacizumab for the manufacture of a medicament for the treatment of renal cell carcinoma in a patient, wherein the treatment includes the administration of the medicament to at least one renal artery of the patient.

In another aspect, embodiments of the present invention encompass the use of an anti-transforming growth factor β monoclonal antibody for the manufacture of a medicament for the treatment of renal cell carcinoma in a patient, wherein the treatment includes the administration of the medicament to at least one renal artery of the patient.

In another aspect, embodiments of the present invention encompass the use of a platelet derived growth factor receptor kinase inhibitor monoclonal antibody for the manufacture of a medicament for the treatment of renal cell carcinoma in a patient, wherein the treatment includes the administration of the medicament to at least one renal artery of the patient.

In another aspect, embodiments of the present invention encompass the use of a mesenchymal stem cell for the manufacture of a medicament for the treatment of acute kidney injury in a patient, wherein the treatment includes the administration of the medicament to at least one renal artery of the patient.

In another aspect, embodiments of the present invention encompass the use of an embryonic stem cell for the manufacture of a medicament for the treatment of acute kidney injury in a patient, wherein the treatment includes the administration of the medicament to at least one renal artery of the patient.

In another aspect, embodiments of the present invention encompass the use of an adult stem cell for the manufacture of a medicament for the treatment of acute kidney injury in a patient, wherein the treatment includes the administration of the medicament to at least one renal artery of the patient.

In another aspect, embodiments of the present invention encompass the use of a stem cell for the manufacture of a medicament for the treatment of late stage kidney disease in a patient, wherein the treatment includes the administration of the medicament to at least one renal artery of the patient.

In another aspect, embodiments of the present invention encompass the use of a growth factor for the manufacture of a medicament for the treatment of acute kidney injury in a patient, wherein the treatment includes the administration of the medicament to at least one renal artery of the patient.

In another aspect, embodiments of the present invention encompass the use of an anti-apoptic for the manufacture of a medicament for the treatment of acute kidney injury in a patient, wherein the treatment includes the administration of the medicament to at least one renal artery of the patient.

In one aspect, embodiments of the present invention encompass systems and methods for treating a renal cell carcinoma in a patient in need thereof. Exemplary approaches can include placing a self-cannulating bifurcated catheter within the abdominal aorta of the patient, where the self-expanding bifurcated catheter includes a first branch extension having a first port and a second branch extension having a second port. Optionally, a bifurcated catheter can be self-adjustable, self-expanding, or self-positioning. Techniques can also include positioning the self-cannulating bifurcated catheter at or near a first renal artery and a second renal artery of the patient, allowing the first branch extension of the self-cannulating bifurcated catheter to expand toward the first renal artery, such that the first port of the first branch extension enters the first renal artery, and allowing the second branch extension of the self-cannulating bifurcated catheter to expand toward the second renal artery, such that the second port of the second branch extension enters the second renal artery. Techniques can also include infusing a fluid agent through the first and second ports of the first and second branch extensions, respectively, and into the first and second renal arteries of the patient, respectively.

Embodiments of the present invention encompass systems and methods for treating a patient that involve, for example, administering an agent to two renal arteries of the patient. The agent can include a stem cell, or a regenerative drug, or an enzyme regulator, or any combination thereof.

For a fuller understanding of the nature and advantages of the present invention, reference should be had to the ensuing detailed description taken in conjunction with the accompanying illustrations.

### DETAILED DESCRIPTION OF THE INVENTION

The FlowMedica Benephit Infusion System is a bifurcated infusion catheter (BIC) that implements targeted renal therapy (TRT) through the localized infusion of prophylactic and therapeutic drugs to, but not limited to, the renal arteries. This catheter is able to rapidly infuse prophylactic or therapeutic drugs directly to either one or both kidneys through the femoral, brachial, or radial access during PPI, surgery, ICU, PCI, TEVAR, PTRA. Unlike with intravenous (IV) delivery, targeted renal therapy (TRT) improves the therapeutic and prophylactic window of drugs in two ways: it increases the local drug concentration in the kidney thus allowing for increased therapeutic efficiency, and it avoids serious systemic and renal side effects associated with intravenous infusion. Since the catheter is well suited for use with various access sites and since it enables simultaneous bilateral infusion with a single catheter, it is viable for numerous clinical specialties and is useful for the treatment of a variety of renal-related diseases. Targeted Renal Therapy opens the door for the treatment of numerous renal diseases such as, but not limited to, AKI, RCC, PKD, CKD, and the overall regeneration of the kidney.

I. Acute Kidney Injury

With regards to AKI, embodiments of the invention encompass the treatment of individuals in the first three R.I.F.L.E. stages: Risk, Injury, and Failure. Depending on the region of AKI the stage of injury may vary. Acute tubular necrosis is a disease of the kidney that can span all stages of R.I.F.L.E., thus the treatment regimens can differ depending on the stage. Relatedly, one or more of these stages or stratifications can apply to glomerulonephritis and interstitial nephritis, for example. Treatment can involve prophylaxis, post-injury administration, and the like. Levels of injury can be based on the measurement of serum creatinine or other biomarkers. According to some embodiments, specific treatments of ATN can be grouped for the prevention of ATN where there is a serum creatinine increase of 50% over baseline creatinine level, a decrease in GFR by 25%, or a urine output below 0.5 ml/kg/h over six hours, and for the treatment of ATN where there is a serum creatinine increase of 100% over baseline creatinine level, a decrease in GFR by 50%, or a urine output below 0.5 ml/kg/h over twelve hours.

Acute Tubular Necrosis can be defined as the death or necrosis of renal tubular cells as a result of either decreased blood flow to the kidneys, nephrotoxic drugs, or both. The decreased blood flow to the kidneys, which can be caused by both extra-renal (systemic hypotension/bleeding) and intra-renal factors (renal vasoconstriction caused by sepsis, HRS, or nephrotoxic drugs), can initiate renal tubular necrosis and apoptosis due to lack of oxygen. This injury can lead to tubular obstruction, tubular back leak, and tubular cast formation. Concurrently, the reperfusion of oxygenated blood in the undamaged tubules can trigger the reduction of ATP/GTP and a series of events leading to DNA damage and finally cell apoptosis. As a result of the renal dysfunction, cytokines can recruit various leukocytes which can cause further oxidative stress and cytotoxic cell destruction. All these factors can lead toward decreased Glomerular Filtration Rate and decreased Kidney Function.

The pathophysiology of acute tubular necrosis pathophysiology may be associated with factors such as ischemic injury, tubular damage due to back leak, obstruction, cast formation, reperfusion injury, and inflammation-induced injury.

In summary, use of a variety of different drugs can be helpful in prevent and treating ischemic injury, reperfusion injury, and concurrent inflammatory injury. The various pathophysiologic mechanisms associated with tubular necrosis can be addressed with a multifaceted therapeutic approach in order to prevent and treat this complex disease. This multifaceted therapeutic approach can include the use of vasodilators, anti-oxidants, free radical scavengers, anti-apoptotic drugs, growth factors, and anti-inflammatory drugs.

Embodiments of the present invention include systems and methods for both preventing and treating tubular necrosis caused by hypoperfusion. To prevent and treat decreased blood flow to the kidney due to extra-renal hypovolemia from shock, hemorrhage, and bleeding, or intra-renal vasoconstriction from sepsis or nephrotoxic drugs, it can be helpful to infuse a vasodilator drug intra-renally. Dopamine dl receptor agonists including, but not limited to, fenoldopam and low dose dopamine; B-type natriuretic peptide receptor A agonists including, but not limited to, nesiritide; A-type natriuretic peptide receptor A agonists including, but not limited to, ularitide and urodilatin; L-type voltage-gated calcium channel blockers including, but not limited to, amlodipine, verapamil and clevidipine; phosphodiesterase-3 and phosphodiesterase-4 inhibitors including, but not limited to, dyphylin, etophylline, theophylline, diethylaminoethyl theophylline; CO-releasing compounds; and prostaglandin 12 receptor agonists including, but not limited to, treprostinil and epoprostenol, all can provide sufficient renal vasodilatation for the prevention and treatment of ischemic injury when infused intra-renally using the bifurcated renal catheter. Calcium channel blockers including, but not limited to, amlodipine, verapamil and clevidipine, can also play a role in preventing Ca2+ injury triggered by ATP reduction to the kidney through the activation of proteases, phospholipases, and cytoskeletal degradation, leading to cell apoptosis.

Tubule cell metabolism may be altered following ischemic acute kidney injury (AKI). ATP depletion can occur in an initiation phase, which can activate a number of oxidative and cell death mechanisms and prime the cell. In an extension phase, prolonged ischemia and reperfusion can urge these pathways to completion. The result can be apoptosis and oxidant injury. Therapeutic approaches can involve inhibition of these pathways, for example by administration of caspase inhibitors, iron chelators, and anti oxidants.

By preventing extra cellular calcium across the vascular cell membrane, calcium channel blockers can prevent the damage caused by the increased levels of Ca2+. The intra-renal infusion of these vasodilators is an effective and safe route of administration for the prevention and treatment of ischemic kidney injury. In comparison, systemic infusion can cause systemic hypotension and thus further exacerbate the ischemia and the damage to the kidney. TRT therapy with vasodilators can be effective as a preventative therapy, and can also be helpful for treating tubular necrosis. An intra-renally delivered agent can be dosed according to the patient's weight. Optionally, the intra-renally delivered agent can be administered for a fixed infusion rate regardless of the patient's weight. In some cases, the means of delivering the agent through the catheter includes an infusion pump.

Embodiments of the present invention include systems and methods for both preventing and treating the tubular necrosis caused by reactive oxygen species (R.O.S.). Reactive oxygen species can play an important role in the pathophysiology of ATN in that they can cause a large portion of the DNA damage that leads to the apoptosis of the tubular cells. A safe and effective method of treating and preventing R.O.S. induced injury to the kidney caused by reperfusion and the inflammatory cascade during acute tubular necrosis includes the intra-renal administration of anti-oxidants and free-radical scavengers.

Glutathione s-transferase activators including, but not limited to, n-acetylcysteine, acetylcysteine, and reduced glutathione; and ascorbic acid have strong anti-oxidant effects and are effective TRT drugs. N-acetylcysteine, an anti-oxidant and vasodilator, when infused intra-renally, helps reduce vasoconstriction in the kidney while countering numerous reactive oxygen species. Other antioxidants are well suited for use with TRT.

TRT with 2-mercaptoethane sulfonate (mesna) defends the kidney against oxidative stress injury to the tubules while avoiding potentially dangerous systemic hypotensive and immunosuppressive adverse effects. Mesna relieves and prevents oxidative tissue stress associated with the inflammatory cascade and I-R injury in ATN. Mesna can induce systemic hypotension and immune suppression when administered intravenously.

The intra-renal infusion of sodium bicarbonate also provides defense against oxidative stress. Sodium bicarbonate can prevent and treat rhabdomyolysis/myoglobinuria induced damage to the kidney by neutralizing the effects of hemoglobin. It can also prevent and treat hyperuricemia-induced kidney injury associated with tumor lysis syndrome by neutralizing the uric acid and preventing the formation of uric acid crystals that block the tubules. Finally it can reduce the damage from numerous other pH-dependent nephrotoxins and contrast agents. Sodium bicarbonate is a pH-neutralizing drug that prevents or inhibits free radical injury by slowing pH dependent radical production and scavenging peroxynitrite and many other R.O.S.'s generated from NO.

Sodium Bi-carbonate can be used to treat acute kidney injury, and can inhibit or prevent hemoglobin induced kidney injury and oxidative stress kidney injury. pH dependent nephrotoxic drugs, such as certain contrast agents, can be neutralized with infusion of sodium bicarbonate.

VHL refers to von Hippel-Lindau protein, HIF hypoxia-inducible factor, TGF-a transforming growth factor a, VEGF vascular endothelial growth factor A, PDGF13 platelet derived growth factor f), EGFR epidermal growth factor receptor, VEGFR2 VEGF receptor 2, PDGFRβ) PDGF receptor f), PTEN phosphatase and tensin homologue, TSC1 and TSC2 tuberous sclerosis complex 1 and 2, FKBPI2 FK506-binding protein 12 kD, mTOR mammalian target of rapamycin complex 1 kinase, eIF4E eukaryotic translation initiation factor 4E, and S6K S6 kinase.

Mutation or inactivation of the von Hippel Lindau (VHL) gene can lead to certain consequences. VHL typically encodes protein (p-VHL) which targets hypoxia-inducible factor (HIF) for proteolysis. VHL inactivation can lead to production of defective p-VHL. Hence, HIF can be up-regulated, and translocated to the nucleus. This can lead to transcription of several genes involved in angiogenesis and tumor growth. Vascular endothelial growth factor (VEGF), platelet-derived growth factor (PDGF), epidermal growth factor (EGF), transforming growth factor (TGF)-α, basic fibroblast growth factor (bFGF), carbonic anhydrase IX (CA IX) or G250, erythropoietin (EPO), and others may be involved. OH refers to hydroxyl group; Ub, ubiquitin; Glut-I, glucose transporter 1; PAI-1, plasminogen activator inhibitor 1.

TRT applied with iron chelating agents including, but not limited to, deferiprone, deferoxamine, apotransferrin and NGAL, prevents and treats free iron, derived from red blood cells and injured cells, triggered production of oxygen species such as superoxide and hydroxyl radicals. Intra-renal infusion of iron chelators versus oral or intravenous administration increases the safety of the drug by avoiding systemic hypotension, potential hepatic fibrosis, and cardiac injury.

TRT therapy with edaravone, an exceptionally effective free radical scavenger and lipid peroxidation inhibitor that are indicated for the treatment of cerebral ischemia can be applied to prevent or treat renal disease.

Intravenous administration of sodium bicarbonate and the various anti-oxidants, especially n-acetylcysteine, typically involve very high doses, which are needed so that the desired effects reach the kidney. Often, such high systemic doses are associated with patient discomfort. Intra-renal infusion techniques are well suited for avoiding this result. Embodiments of the present invention encompass treatments that involve the administration of various combinations of these agents. These intra-renally delivered agents can be dosed according to the patient's weight. Optionally, the intra-renally delivered agents can be administered for a fixed infusion rate regardless of the patient's weight. In some cases, the means of delivering the agent through the catheter includes an infusion pump.

Embodiments of the present invention include systems and methods for both preventing and treating the tubular necrosis caused by cell apoptosis. Cell apoptosis is a series of programmed cell death due to DNA damage caused by the various paths of injury. After DNA damage, apoptosis occurs through the release of various enzymes that participate in DNA repair and programmed cell destruction.

TRT applied with minocycline provides exceptional prevention and treatment of cell apoptosis because of its proven anti-apoptotic and anti-inflammatory effects. It is able to prevent cell apoptosis by reducing the mitochondrial release of proteins and enzymes such as cytochrome c, p53, and bax, that regulate cell repair and cell cycle, and it is especially effective if infused pre-ischemic or pre-toxic injury to the kidney. Intra-renal infusion of minocycline is a safer system of administration because when infused intravenously it can cause immune-suppression and then leaves the body susceptible to systemic disease and infections. TRT allows for a higher more concentrated and localized dose.

TRT infusion of recombinant human erythropoietin can prevent or treat apoptosis, decrease R.O.S. damage, and vasodilate the kidney. Erythropoietin can induce heme oxygenase-I, a rate limiting enzyme that catalyzes the degradation of heme to iron, carbon monoxide (CO), which causes vascular vasodilatation, and biliverdin, which protect the kidney from oxidative stress by restoring the endothelial and nitric oxide synthase levels and increasing anti-apoptotic proteins. Pre-treatment with erythropoietin can have structural and functional protective effects on the kidney, and inhibit apoptosis through the transcription factor NF-KB. Intra-renal infusion of erythropoietin provides a safe alternative to intravenous infusion, as systemic infusion requires upper dosing for equal efficiency. TRT with erythropoietin, for example, is advantageous due to a decreased risk of tumor growth, hypotension, and numerous other extrarenal adverse effects.

TRT infusion of heat shock proteins (HSP) provides a cytoprotective protein of the tubule cells. Heat shock proteins help promote the survival of tubule cells by inhibiting apoptosis in addition to helping restore tubule cell function by guiding the cells through repair. Currently, HSP therapy has not been actively pursued in humans systemically because of worries of extrarenal immunogenicity and adverse effects. Administered intra-renally, however, a higher dosage can be given without effecting immune response throughout the body.

TRT infusion of hepatocyte growth factor (HGF) and fibroblast growth factor provides anti-apoptotic effects by helping facilitate cell repair. Tubule cells that have survived injury are able to dedifferentiate, proliferate, and then transdifferentiate back into tubule cells after acute kidney injury. Growth factors can be reno-protective by increasing cell proliferation and thus increasing cell repair after injury. Direct injection into the kidney is advantageous with these growth factors in order to facilitate the repair.

Tubule cell structure can be altered after ischemic AKI. An initiation phase can cause to sublethal injury, which may be characterized by loss of brush borders and disruption of cell polarity and the cytoskeleton. Alleviation at this stage of the injury can lead to complete recovery. If unalleviated, the injury may progress to an extension phase. Cell death, desquamation, luminal obstruction, and an inflammatory response may ensue. Inhibition of apoptosis and inflammation at this stage can be therapeutic. Tubule cell death and restoration are typically balanced in the maintenance phase. This can occur as a result of viable tubule cell differentiation, stem cells, progenitor cells, and the like. Acceleration of the endogenous regenerative process can be beneficial, and speed recovery.

TRT with caspase inhibitors is useful for preventing or treating ATN during the late stages of ischemic injury. TRT with caspase inhibitors is useful as a treatment after the onset of ischemic injury for the prevention or treatment of post-ischemic cell apoptosis. Caspases are proteases that initiate and execute cell apoptosis. As a result, caspase activation is widely considered the final step in the apoptosis of cells.

TRT with PARP inhibitors is helpful to prevent or inhibit further damage in the post-ischemic injury setting by sustaining energy transfer between cells and preventing the additional release of R.O.S. PARP inhibitors can prevent or treat cellular injury caused by the over activation of PARP. PARP is an enzyme that plays a role in DNA repair; however, when it is over activated, it can trigger the reduction of intercellular NAD+, a coenzyme that plays an important role in the carrying of electrons, and ATP, a nucleotide that is the molecular currency of energy transfer between cells. Also PARP triggers the release of reactive oxygen species.

Intra-renal administration of Guanosine triphosphate (GTP) is helpful for preventing and treating ischemic injury to the kidney before or during injury. (GTP) is similar to ATP in that it plays an important role in the transfer of energy; more specifically GTP is an activator of substrates in metabolic reactions and is used as a source of energy for protein synthesis. During the inflammatory cascade, GTP and ATP are depleted and as a result, DNA damage occurs which finally leads to cell death.

TRT with JNK inhibitors can prevent or treat kidney injury during the inflammatory cascade. JNK inhibitors block the activity of C-jun N-terminal kinases which turn on specific cell death genes and cytokines for the initiation of cell apoptosis and inflammation. The JNK pathway regulates the expression of cytokines, growth factors, and cell death genes that have large roles in inflammatory diseases and apoptosis.

TRT with pifithrin-a (p53 inhibitor) is helpful for preventing or treating renal disease. Pifithrin-a delays the onset of cell death by inhibiting p53, a transcription factor that regulates the cell cyele and programmed cell death. As a result p53 inhibitors can allow the damaged DNA to repair itself through the bodies repair mechanisms before the tubule cell is destroyed.

In sum, TRT with caspase inhibitors, Poly ADP-Ribose Polymerase (PARP) inhibitors, C-Jun N-terminal kinase (JNK) inhibitors, and Guanosine and Pifithrin-a inhibitors is helpful in preventing or treating renal-related disease. These agents play a role in preventing or inhibiting the initiation and execution of programmed cell death associated with reperfusion and inflammation.

Human ischemic AKI can involve major apoptotic pathways. Activation of plasma membrane Fas receptor may be needed for the extrinsic pathway. Activation of caspase 8 can be the result of signal transduction via FADD. Translocation of Bax to the mitochondria may be needed for the intrinsic pathway. Release of cytochrome c and activation of caspase 9 can occur through pores thus formed. Bid activation can provide cross-talk between these pathways. Bax can be activated by p53-dependent pathways, and Bax activation can be prevented in normal cells by Bcl2 and Bcl-xL. Caspase 3 can be activated by both caspases 8 and 9, and can initiate final morphologic cascades of apoptosis. Inhibition of these pathways can be beneficial for treating acute renal failure.

Microvasculature alterations and inflammation can occur ischemic AKI. In the extension phase endothelial injury can lead to intense vasoconstriction, microvascular sludging, and micro-vascular congestion with leukocytes. Inflammatory mediators and reactive oxygen species can be produced by activated leukocytes. Hence, tubule cell damage can be potentiated. Tubule cells can show a maladaptive response by generating cytokines and chemokines. Hence, inflammation can be increased. PMN, polymorphonuclear leukocyte, Thl, T-helper 1 cell can be involved. Ischemic AKI therapy can encompass techniques that modulate the inflammatory response.

The likelihood of acute renal failure during septic shock can be decreased by administration of arginine vasopressin (AVP) and hydrocortisone (50 mg every six hours for seven days). Such treatment can be effective for pressor-resistant hypotension. In patients with sepsis, early directed resuscitation can prevent or inhibit the progression from prerenal azotemia to acute tubular necrosis. Incidence of acute renal failure, multiple-organ dysfunction syndrome, and death can be decreased by maintenance of blood glucose levels below 145 mg per deciliter (8.0 mmol per liter). Disseminated intravascular coagulation with glomerular and microvascular thrombi can be decreased by activated protein C, thus decreasing mortality. Inhibition is shown by T bars.

An intra-renal infusion of any desired combination of these apoptosis inhibitors provides a precise, concentrated, and safe therapy for apoptosis associated with acute tubular necrosis. Depending on the stage of apoptosis, the drugs that can be infused intra-renally may vary, as well as the dosage and the timing of therapy. The effects of these inhibitors and anti-apoptotic drugs may be dependent on their localized injection to the area where injury has or will occur. These intra-renally delivered agents can be dosed according to the patient's weight. Optionally, the intra-renally delivered agents can be administered for a fixed infusion rate regardless of the patient's weight. In some cases, the means of delivering the agent through the catheter includes an infusion pump.

Embodiments of the present invention include systems and methods for both preventing and treating the inflammatory damage in ATN. Much of the kidney injury associated with tubular necrosis is often due to the inflammation that occurs as a result of the tubular damage and re-oxygenation of tubules. Ischemic acute kidney injury can lead to the endothelial's over expression of adhesion molecules that promote leukocyte interactions with the endothelial and platelets, and the endothelial damage is then supplemented by the generation of maladaptive pro-inflammatory and chemotactic cytokines that recruit more leukocytes to the tubules through the expression of selectins. This can further amplify the damage to the tubules. TRT with various agents can prevent, inhibit, or counter the inflammation and the processes that lead to leukocyte recruitment. TRT therapy may involve administration of platelet aggregation inhibitors and platelet adhesion inhibitors including, but not limited to, tirofiban hydrochloride; neutrophil inhibitors including, but not limited to, neutrophil chemotaxis inhibitor; P-38 MAP kinase inhibitors; and anti-inflammatory drugs including, but not limited to, adenosine receptor agonist, peroxisome proliferator-activated receptors (PPAR), C5a receptor antagonist, IL-I0, IL-6 antagonist, statins, α-Melanocyte stimulating hormone, which have specialized roles in either deactivating the inflammatory cascade or preventing signaling of white bloods cells. A combination of one or more of these drugs provides protection against the inflammatory cascade when infused during TRT.

TRT with platelet aggregation inhibitors such as tirofiban hydrochloride (Aggrastat®) and platelet adhesion inhibitors is helpful in preventing or treating injury due to inflammation. The mechanism of action may involve prevention or inhibition of the initial aggregation of platelets and adhesion of platelets to the vascular walls which cause the leukocyte response. TRT therapy can also involve administration of P-selectins. These compounds can play a role in recruiting leukocytes to the site of injury during inflammation, are activated by platelets during injury. Intra-renal infusion of these agents can avoid inhibition of platelets in the other areas of the body. If infused intravenously, adverse effects include excessive bleeding which can be detrimental to the body. Immunosuppressant drugs when infused systemically can put the body at great risk of infection and other injuries.

TRT with neutrophil chemotaxis inhibitors provide early support for countering or inhibiting inflammation in the kidney. Neutrophils are recruited to the kidney by selectins because of the generation of cytokines. Chemotaxis directs the movements of cells and molecules in the body, and thus a neutrophil chemotaxis inhibitor can prevent the movement of neutrophils to the area of inflammation after their recruitment by selectins. As with platelet aggregation inhibitors, neutrophil chemotaxis inhibitors can be infused intra-renally through TRT in order to prevent or inhibit immunosuppressant damage throughout the rest of the body. Alternatively, inhibition of pro-inflammatory cytokines such as TNF-α and interlukin 1-13, selectively within the kidney, by an intra-renally delivered P38 kinase inhibitor can reduce neutrophil recruitment to the injured kidney without adversely affecting other necessary inflammatory processes in the body.

TRT with adenosine presents a safe and effective therapy for cases of inflammation, including cases of rapid-onset inflammation. Adenosine receptor agonists can play a role in preventing or inhibiting tubular injury due to the inflammatory cascade by inactivating it. Adenosine can prevent or inhibit the immune response and as a result can prevent or inhibit the various free radical, ROS, and other detrimental enzymes and compounds from being released in the kidney. In addition to immune response inhibition, it can also be responsible for vascular regulation, and when administered can stimulate vasodilation of the kidney. As with other vasodilators and immunosuppressants, the intravenous infusion can lead to hypotension related problems and also immune response related problems.

TRT with peroxisome proliferator-activated receptors (PPAR) can have protective effects on the kidney by preventing or inhibiting the initiation of inflammatory white cell recruitment and signaling. PPARs are transcription factors that can play a role in the prevention or inhibition of inflammation by regulating glucose and lipid metabolism. Pre-treatment of PPAR can help reduce nephrotoxic induced kidney injury, while also suppressing NF-KB activation, cytokine and chemokine expression, and neutrophil recruitment into the kidney.

TRT with C5a antagonists is helpful in treating or preventing renal-related disease. C5a is a chemoattractant that is expressed in the kidney's tubule epithelial cells and interstitial macrophages that recruits leukocytes such as neutrophils, T-cells, and monocytes during injury. Due to its anti-inflammatory effects, C5a antagonists provide protection of renal dysfunction in cases of ischemic-reperfusion injury and sepsis.

TRT with IL-6 antagonists or other anti-inflammatories is helpful in treating or preventing renal-related disease. IL-6 antagonists are anti-inflammatories that reduce structural and functional consequences of ischemic injury by countering IL-6, a pro-inflammatory cytokine; IL-I0, a powerful anti-inflammatory which inhibits the production of maladaptive cytokines by Thl cells; or a-Melanocyte stimulating hormone, an anti-inflammatory cytokine that inhibits the maladaptive gene activating that causes inflammation and cytotoxic injury to the kidney, will counter certain elements of the inflammatory cascade. These anti-inflammatory drugs can play specific roles in inhibiting the activation and damage due to cytokines and the inflammatory cascade following their expression. Intra-renal administration of these agents is beneficial because systemic administration can lead to adverse immune related side effects.

As with apoptosis, the inflammatory cascade is very complex. TRT therapy can involve the administration of a combination of one or more agents to prevent or inhibit damage associated with various pathways of the inflammatory cascade. Anti-inflammatory drugs can run the risk of causing serious immune suppression and various adverse effects when systemically infused as a cocktail. TRT with these drugs provides a safe and effective route of administration to quickly prevent or treat kidney injury and systemic infections and bone marrow suppression in cases of ATN, AIN, and AGN. These intra-renally delivered agents can be dosed according to the patient's weight. Optionally, the intra-renally delivered agents can be administered for a fixed infusion rate regardless of the patient's weight. In some cases, the means of delivering the agent through the catheter includes an infusion pump.

Embodiments of the present invention include systems and methods for treating Acute Kidney Injury induced by sepsis. Sepsis is a serious infection that induces vasoconstriction and inflammation in the kidney and has emerged as one of the greatest causes of acute kidney injury. TRT therapy is helpful for preventing or inhibiting sepsis-induced acute tubular necrosis. For example, such TRT therapy may include the intra-renal infusion of activated protein c (APC), which can prevent or inhibit inflammation and apoptosis of kidney cells. APC is an anticoagulant and can prevent or treat apoptosis and inflammation by inhibiting the activation of leukocytes in the kidney. Related TRT therapy can also involve intra-renal infusion of ethyl pyruvate, optionally in combination with APC. TRT with ethyl pyruvate, an anti-oxidant and free radical scavenger, can help to prevent or treat reactive oxygen species injury to the kidney. TRT therapy can also include the intra-renal infusion of corticosteroids including, but not limited to, hydrocortisone and methylprednisolone, optionally in combination with APC. Intra-renal administration of corticosteroids can inhibit or prevent inflammatory action in the kidney. Corticosteroids are immunosuppressive agents that are safe and effective when infused intra-renally. TRT with insulin can help prevent hyperglycemia and counter the insulin resistance in the body. In addition, intra-renal infusion of insulin can be helpful in treating or ameliorating endothelial dysfunction and subsequent hyper-coagulation and dyslipidemia. These intra-renally delivered agents can be dosed according to the patient's weight. Optionally, the intra-renally delivered agents can be administered for a fixed infusion rate regardless of the patient's weight. In some cases, the means of delivering the agent through the catheter includes an infusion pump.

II. Acute Glomerulonephritis/Acute Interstitial Nephritis

Embodiments of the present invention encompass systems and methods for treating the rapid inflammation associated with acute glomerulonephritis (AGN) and acute interstitial nephritis (AIN). Intra-renal infusion with immunosuppressant agents can be helpful in preventing, inhibiting, or decreasing rapid inflammation occurring in the glomeruli or interstitium which may be associated with AGN or AIN.

AGN and AIN can be caused by an immune-mediated inflammatory response. Acute Interstitial Nephritis (AIN) is a leading cause of AKI, accounting for 10% of all cases. AIN is a rapidly developing inflammation that occurs in the interstitium due to immune-mediated tubulointerstitial injury. It is usually caused by adverse reactions to drugs, infections, and other causes. In fact, several types of glomerulonephritis result in inflammation of the interstitium. This inflammation is detrimental to the kidney and quickly leads to the deterioration of kidney function. Like AIN, Glomerulonephritis (AGN) is also a rapidly developing inflammation; however, this inflammation occurs in the glomerulus of the kidney. AGN represents a set of renal diseases in which an immunological reaction causes inflammation of the glomeruli. The glomerular inflammation is caused either by proliferative damage or non-proliferative damage associated with the immunological reaction.

Embodiments of the present invention encompass TRT techniques for treating inflammation of the interstitium and glomeruli associated with these conditions. Often, such TRT intra-renal infusions include the administration of immunosuppressant drugs including, but not limited to, azathioprine sodium succinate, methylprednisolone, hydrocortisone, and rituximab. Direct renal delivery of these agents can be effective at decreasing the inflammation without causing systemic immunosuppression.

TRT therapy for AGN or AIN includes intra-renal delivery of corticosteroids, including, but not limited to, hydrocortisone and methylprednisolone, and non-steroidal immunosuppressant drugs including, but not limited to, azathioprine. Direct administration of corticosteroids and non-steroidal immunosuppresants into the renal arteries can be helpful for preventing or inhibiting interstitial nephritis, glomerulonephritis, and other inflammatory renal diseases. These immunosuppressant agents can decrease inflammation and thus protect the kidney from damage. Hydrocortisone and methylprednsilone are especially potent anti-inflammatory steroids with less water and salt retention effects than some other agents, making them effective and safe. Systemic administration often lowers the body's defenses to other infections and diseases. This unwanted side effect can be increasingly deleterious when a patient requires extensive therapy over a long period of time. In cases of rapid onset of inflammation, where inflammation reaches its peak in a short span and treatment is needed or desired immediately, the TRT infusion of corticosteroids or azathioprine is a helpful technique to quickly decrease inflammation. TRT infusion is also useful in the prevention or inhibition of rapidly progressive glomerulonephritis, an inflammation of the glomeruli where loss of kidney function occurs in a period of weeks to months.

TRT with a CD20 receptor antagonist provides a quick anti-inflammatory response to the rapid inflammation while also preventing or avoiding systemic and renal injuries due to potential adverse effects. Cluster of Differentiation 20 (CD20) receptor antagonist, including, but not limited to, rituximab, is an antibody that targets a receptor (CD20) on B-cells, which induces lysis through various mechanisms, and works with elements of the immune system to kill CD20+ B-cells, and thus prevents or inhibits specific disorders, including ANCA associated vasculitis and lupus nephritis, associated with glomerulonephritis. Since CD20 receptor antagonist causes immunosuppression, hypotension, and various other cardiac adverse effects, it can be dangerous to administer it systemically.

TRT therapy can also include direct renal administration of numerous other drugs indicated for specific forms of nephritis of either the glomeruli or the interstitium. These drugs contain antibodies and inhibitors that target the specific causes and mechanisms of the inflammation associated with these disorders.

TRT infusion therapy with platelet derived growth factor receptor beta kinase inhibitor can be helpful to prevent or inhibit the growth of renal tumors and the progression of renal cell carcinoma (RCC). Platelet derived growth factor receptor beta kinase inhibitor is a monoclonal antibody that inhibits mechanism of excess platelet derived growth factor. Platelet derived growth factor plays a role in the inflammation of the kidney associated with IgA nephropathy, the most common form of glomerulonephritis. Platelet derived growth factor-beta is also expressed during renal cell carcinoma due to the up regulation of HIF. TRT therapy can also include infusion of Levofloxacin (Levaquin®). Levofloxacin is a bactericidal drug that inhibits the actions of gyrase, a bacterial enzyme that is necessary for the execution of DNA formation and replication. Levofloxacin prevents or inhibits bacterial cells from reproducing and thus is able to fight the bacterial infection associated with pyelonephritis.

TRT with these immunosuppressant drugs provides a safe and effective route of administration to quickly prevent or inhibit kidney injury due to inflammation of the glomeruli or interstitium without leaving the body vulnerable to infections and bone marrow suppression. These intra-renally delivered agents can be dosed according to the patient's weight. Optionally, the intra-renally delivered agents can be administered for a fixed infusion rate regardless of the patient's weight. In some cases, the means of delivering the agent through the catheter includes an infusion pump.

III. Renal Cell Carcinoma

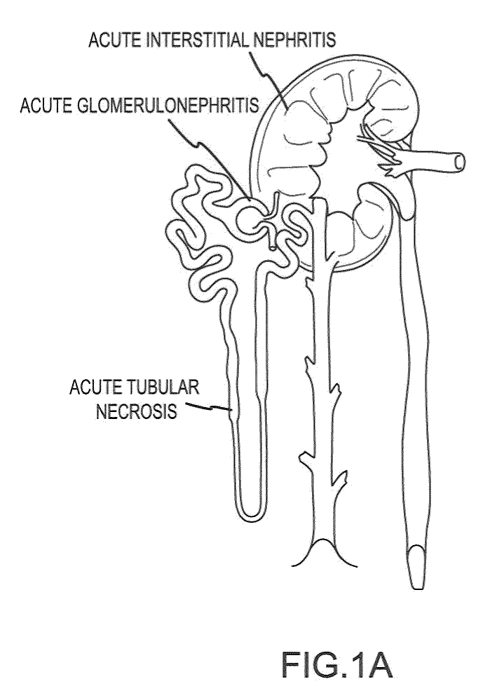

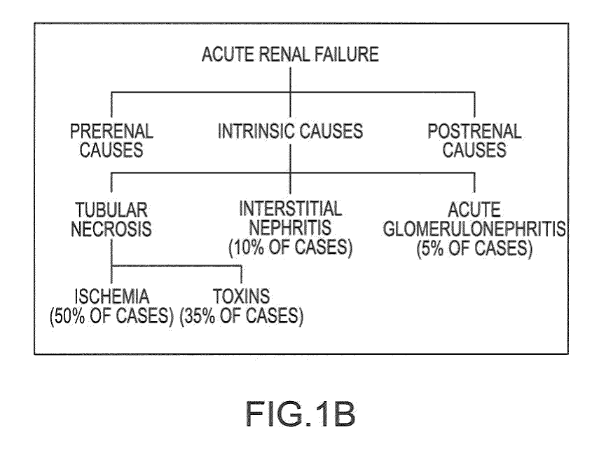

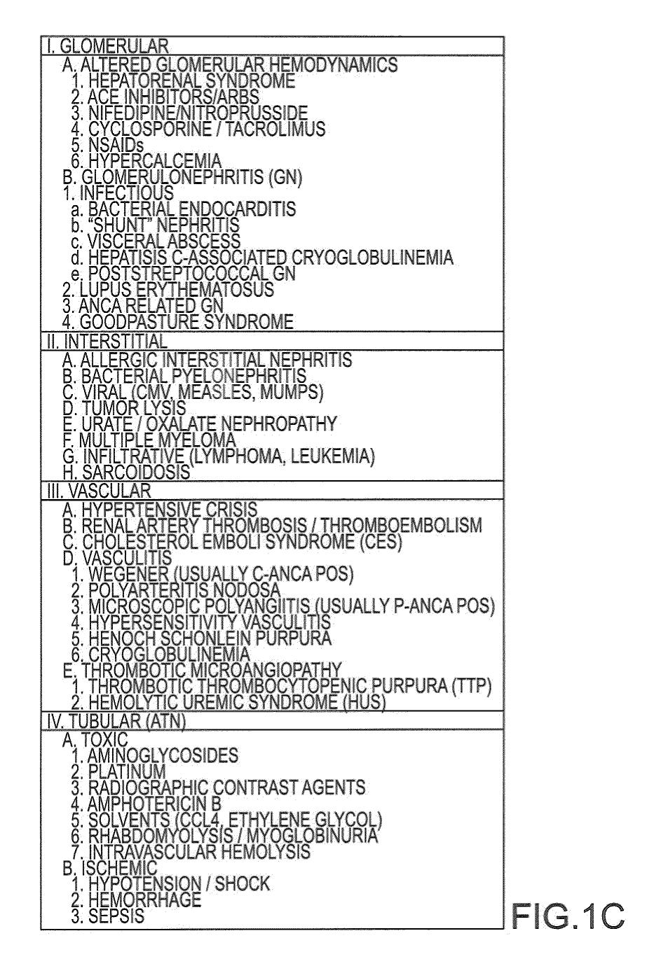

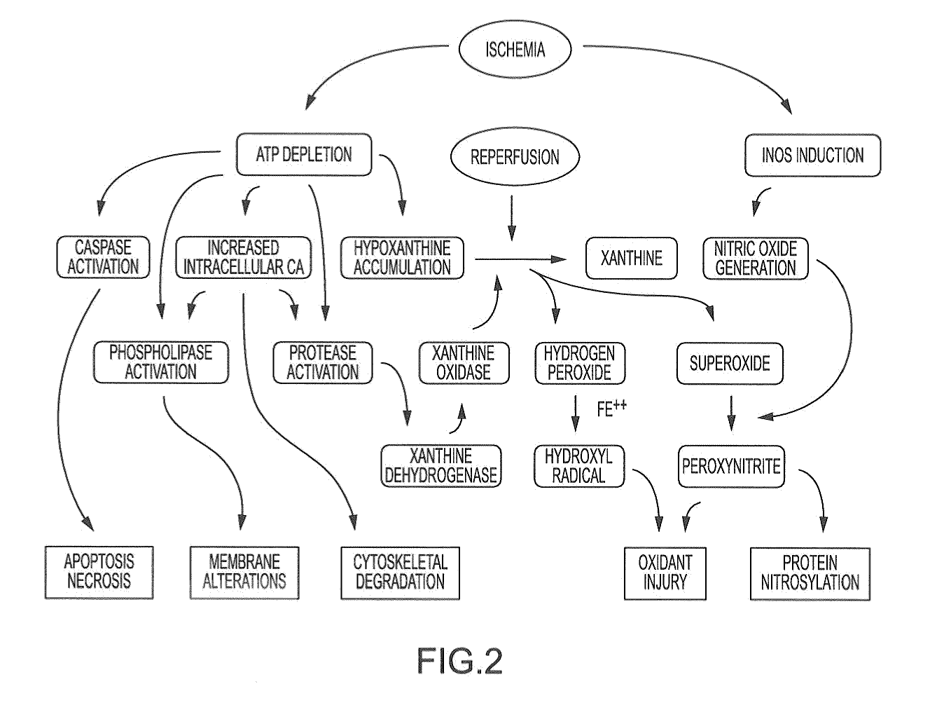

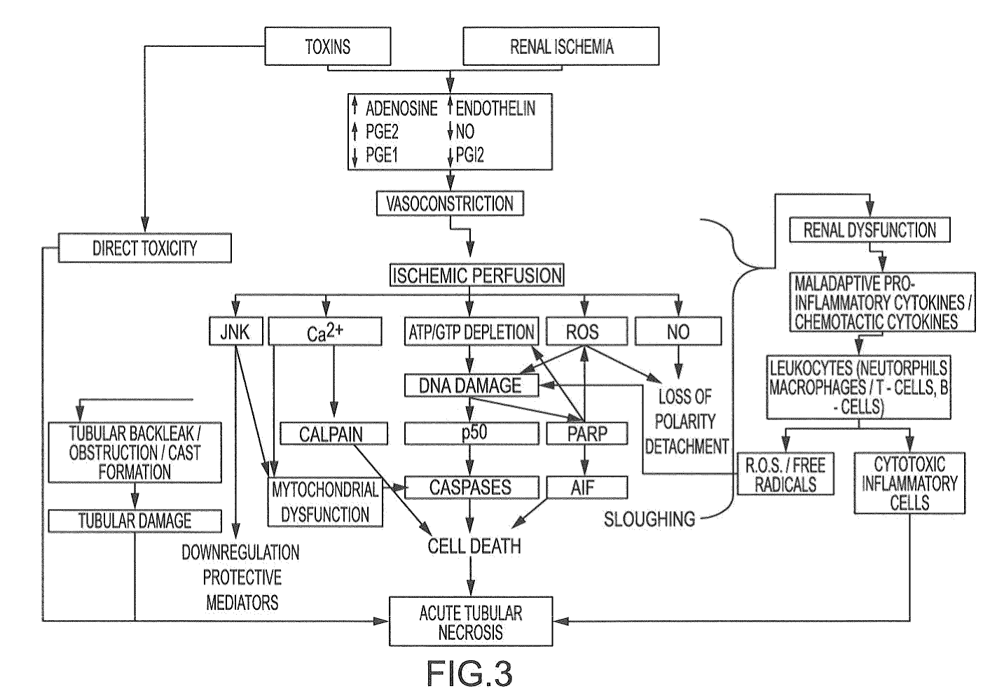

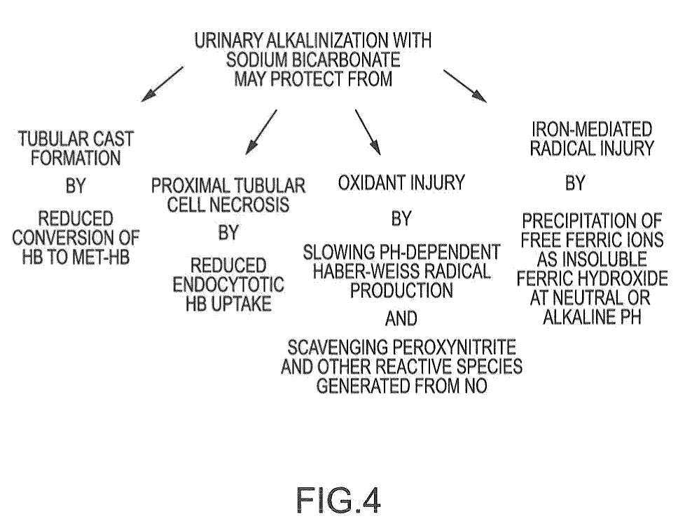

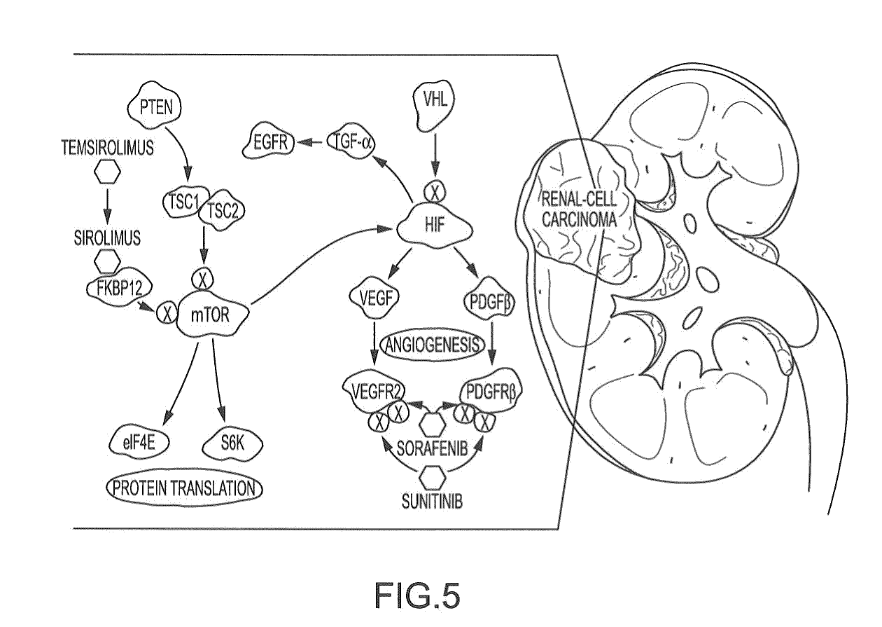

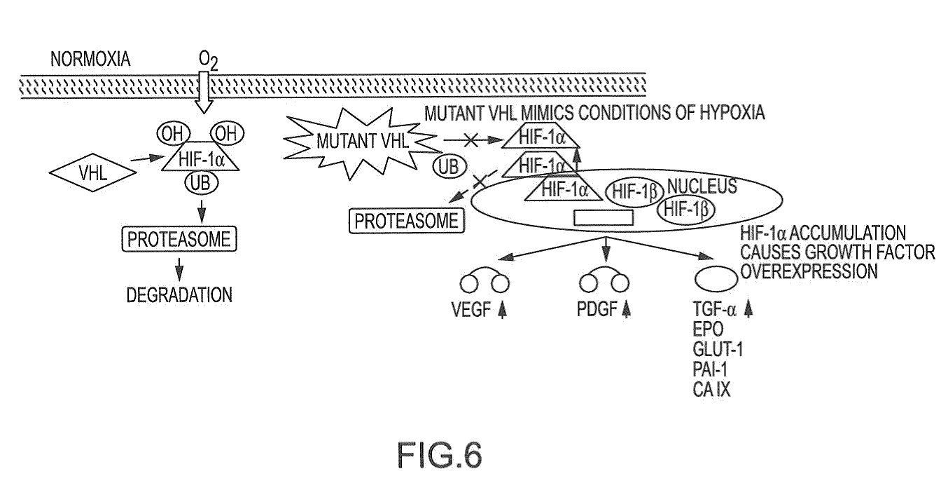

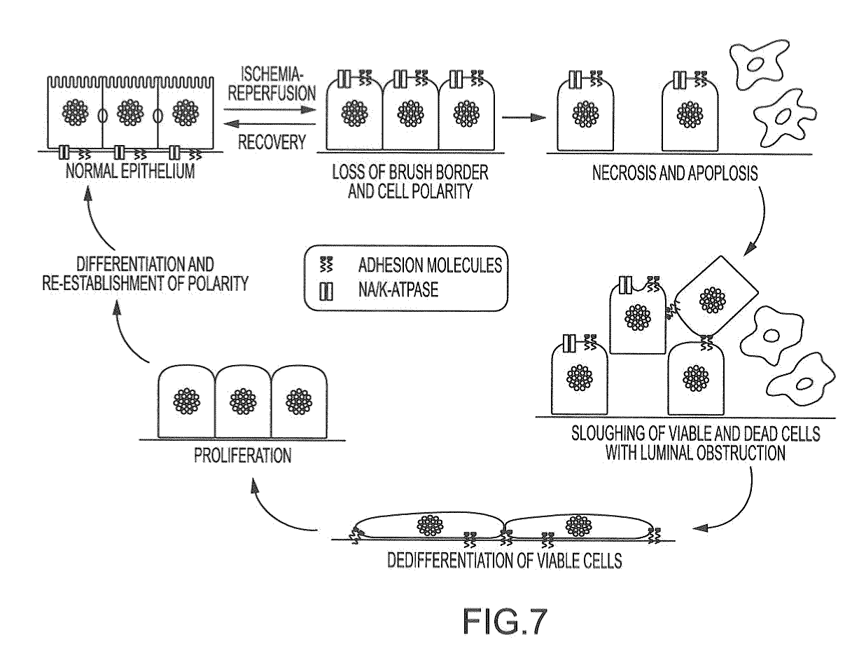

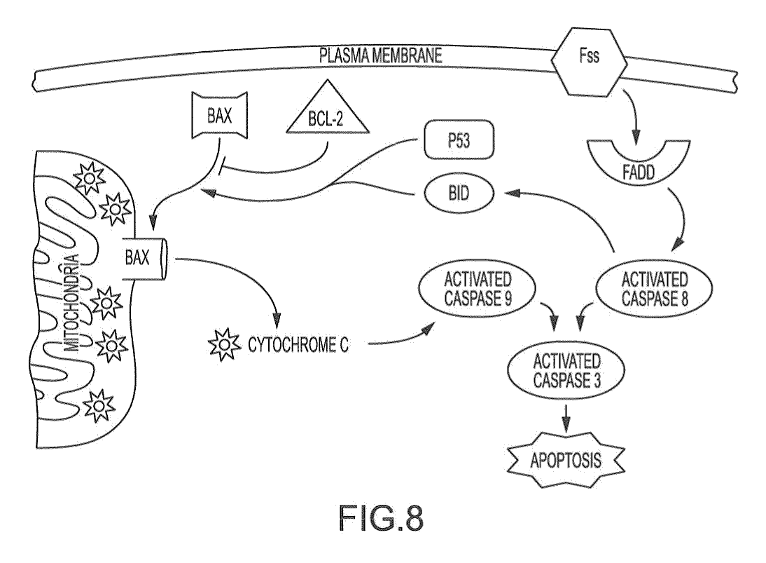

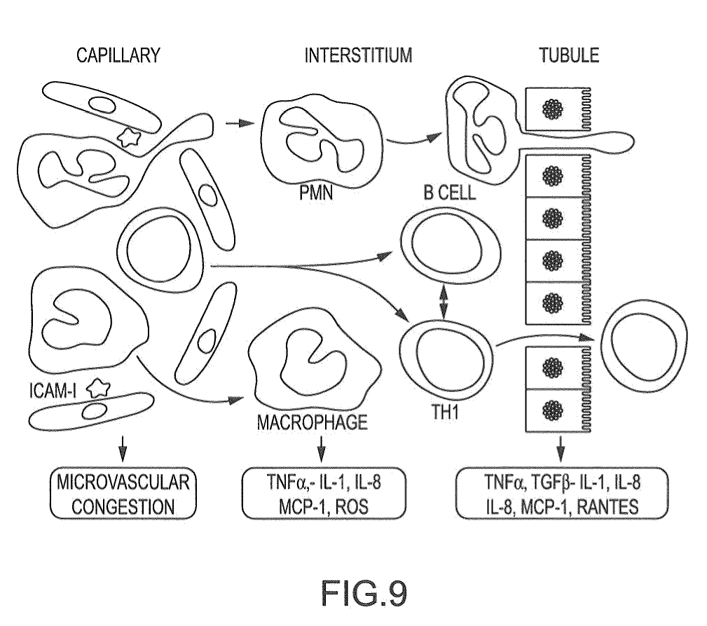

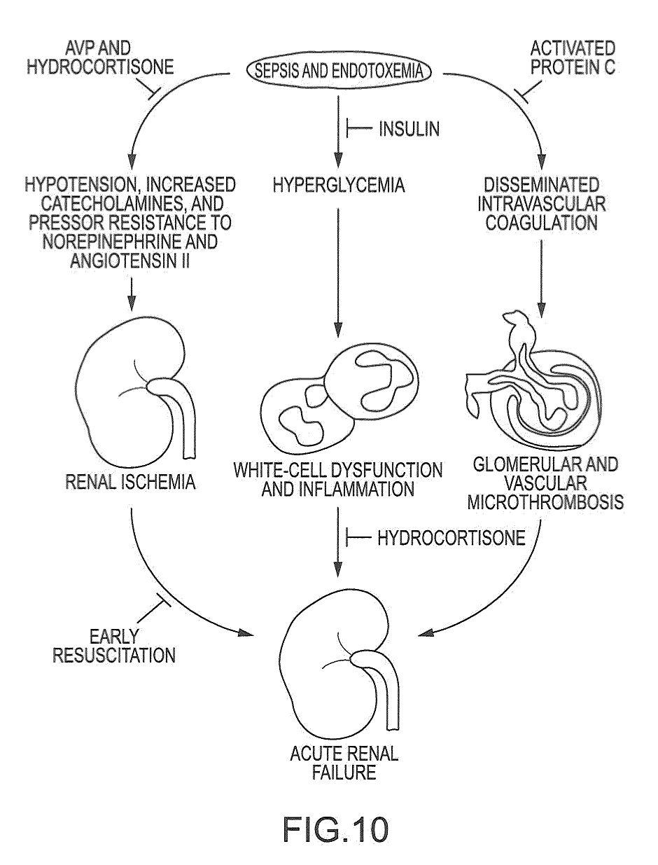

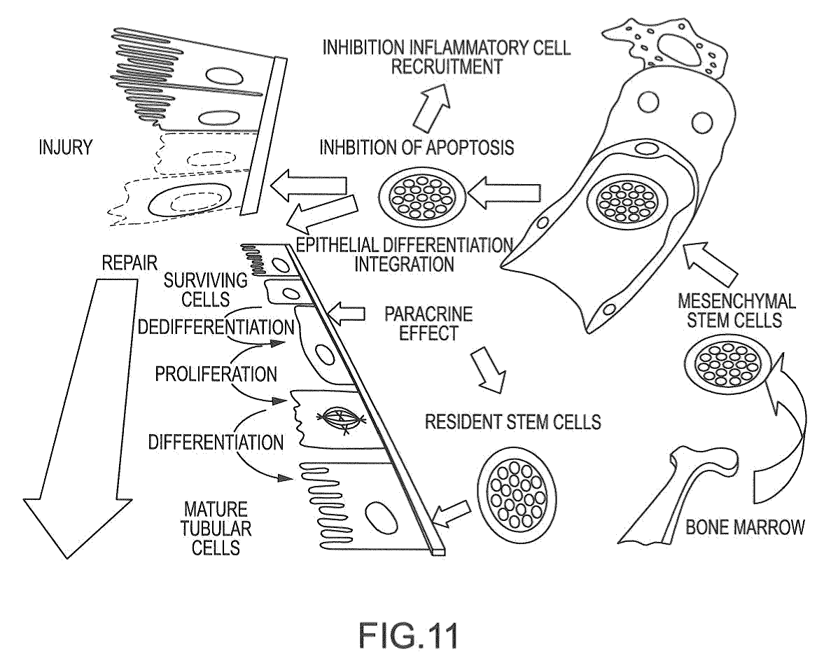

TRT infusion with anti-renal cell carcinoma drugs provides a quick and effective method of removing residual cancer cells after surgery. See FIGS. 9 and 10. Renal cell carcinoma (RCC) is a cancer of the kidney where cancerous cells are found in the tubules of the kidney. RCC stems from the loss of VHL tumor suppressor gene, a gene that targets HIF (hypoxia-inducible factor). As a result HIF proteins are up-regulated throughout the kidney; they then enter the nucleus of kidney cells and stimulate the expression of genes such as VEGF, PDGFβ), and TGF-a that play integral roles in tumor growth. In the United States, an approximately 39,000 new cases of RCC and 13,000 deaths were predicted to occur in the year of 2006. In addition, Renal Cell Carcinoma is the 7th leading malignant condition amongst men and the 12th leading malignant condition amongst women, making it easily the most common form of Renal Cancer, accounting for around 85% of the cases. Although surgery is often the preferred treatment for this cancer, many patients experience a recurrence after surgical treatment; thus there is a pressing need for effective drug therapy. Chemotherapy is typically not an option for treating RCC. However, TRT infusion with anti-renal cell carcinoma drugs is a quick and effective method of removing or treating residual cancer cells after surgery.

TRT therapy with interleukin-2, optionally as a side drug in a cocktail with other drugs, is helpful in preventing or inhibiting RCC. Intravenous high dose interleukin-2 therapy has been proposed for advanced renal cell carcinoma, and is thought to involve sending messages to the immune system in order to stimulate the body's production of T cells that target and kill renal cancer cells. Although it has been proposed as a treatment, the majority of patients do not experience the benefits. This can be attributed to its systemic administration. In some cases, treatment of RCC with interleukin-2 may require high-dose therapy, and systemic administration of interleukin-2 at high doses can be extremely toxic. TRT with interleukin-2 allows for high doses without the dangerous side effects associated with IV administration.

TRT therapy with interferon alpha is helpful in preventing or inhibiting RCC. Interferon Alpha is an immunomodulatory cytokine which is indicated for the treatment of metastatic renal cell carcinoma. Interferon alpha it thought to have three mechanisms: it signals white blood cells to attack the cancerous cells, it inhibits the replication of cancerous and viral cells (tumor growth), and it decreases the blood flow to the cancerous cells. As is the case with interleukin-2, the majority of patients do not experience the benefits when administered systemically.

TRT therapy also encompasses intra-renal administration of drugs that target the TGF-a, mTOR signal transduction, and VEGF pathways. Such techniques can provide treatment of RCC by either killing the cancer cells or preventing or inhibiting the signaling, and expression of growth factors that lead to further tumor growth, or both. Hence, TRT can include the intra-renal administration of one or more of these agents, including without limitation Bevacizumab, Temsirolimus, Ispinesib, and anti-TGF beta monoclonal antibodies.

Bevacizumab is a recombinant humanized antibody to vascular endothelial growth factor (VEGF) which causes cell proliferation and rapid growth of blood vessels leading to tumor formation. TRT with Bevacizumab can be helpful in treating metastatic renal cell carcinoma. Temsirolimus is an mTOR inhibitor that regulates cell growth and angiogenesis by up-regulating and down-regulating various proteins such as HIF-I. HIF-I is a transcription factor that up regulates the expression genes such as VEGF and PDGF13, two growth factors that regulate cell growth and, in the case of RCC, stimulate tumorigenesis. TRT with Temsirolimus can provide improvements in survival. TRT with Bevacizumab, Temsirolimus, or both can provide a safe and efficient treatment by killing the remaining tumor cells and preventing the growth of new ones. TRT with Ispinesib, a novel mitotic kinesin inhibitor, can disrupt the cell cycle of the cancerous cells in the kidney and thus lead to their cell death in a quick and efficient manner. Mitotic kinesins are family of cytoskeletal enzymes that are necessary for the formation and function of mitotic spindles. Inhibition of these enzymes causes the disruption of cell cycle and cell division, leading to their death. TRT with anti-TGF beta monoclonal antibodies can prevent or inhibit the growth and division of cancer cells. TGF monoclonal antibodies are over expressed due to the build up of hypoxia-induced factor (HIF) in renal cell carcinoma and as a result help facilitate the growth, progression, and migration of cancer cells in the kidney.

Most renal cell carcinoma drugs often cause serious adverse side effects when upper-dosed systemically. These side effects include, tachycardia, hypotension, and immunosuppressive adverse effects including, but not limited to, thrombocytopenia, leukopenia, and neutropenia. When infused intra-renally with targeted renal therapy, these drugs can be upper dosed, in order to increase the amount of tumor death and decrease the number of treatments, without risking adverse effects. A multifaceted targeted renal therapy with drugs that both inhibit the growth factors, inhibit the expression of HIF and mTOR, and also kill the residual cancer cells can be helpful to treat RCC. These intra-renally delivered agents can be dosed according to the patient's weight. Optionally, the intra-renally delivered agents can be administered for a fixed infusion rate regardless of the patient's weight. In some cases, the means of delivering the agent through the catheter includes an infusion pump.

IV. Stem Cell Targeted Renal Therapy

TRT with mesenchymal stem cells and other stem cell modalities can be helpful in treating renal-related disease. Stem cell therapy via intra-renal administration can be used to facilitate the repair and regeneration of renal cells that have been damaged or have died as a result of kidney injury. TRT methods of embryonic and adult stem cell injection can involve the in-vitro expansion and injection of mesenchymal stem cells, which can lead to the prevention and reversal of AKI. These mesenchymal stem cells can prevent apoptosis of tubular cells and inhibit the recruitment of inflammatory cells, transdifferentiate into epithelial cells, and also through the paracrine effect can stimulate the dedifferentiation of surviving tubular cells, proliferation, migration, and finally differentiation into epithelial cells. There are problems with intravenous stem cell therapy that are solved with the direct renal administration of stem cells. Relatedly, to prevent or inhibit the immune response to foreign stem cells, immunosuppressant drugs, including, but not limited to, corticosteroids, can be infused intra-renally after stem cell infusion to prevent or inhibit the leukocyte response.

MSCs can play a role in the repair of acute tubular injury. Derived from bone marrow, MSCs may enter the circulation and reach the sites of tissue injury. For example, MSCs can be recovered from bone marrow, expanded in vitro, and administered for therapeutic purposes. MSCs can inhibit apoptosis of tubular cells and recruitment of inflammatory cells, thus benefiting patients with ARF. MSCs may transdifferentiate into mature epithelial cells. MSCs can have a paracrine effect on tubular cells surviving injury by stimulating dedifferentiation, proliferation, migration and eventually re-differentiation into mature epithelial cells. Administered MSCs can modify the microenvironment by allowing expansion and differentiation of resident SCs that may contribute to the repopulation of injured tubules.

V. Additional Approaches

Targeted Renal Therapy (TRT), which can involve the direct delivery of therapeutic agents to the kidneys via the renal arteries, offers a way to increase the therapeutic windows of certain drugs that may have a beneficial renal effect in AKI. The benefits of TRT are significant. Delivery of agents directly to the kidneys via the renal arteries increases the local dose of the agent over that which could be achieved via systemic (intravenous, IV) delivery, as the dilution by the blood is significantly less. Delivery directly to the kidneys can take advantage of renal first-pass, in which the kidney can clear a substantial portion of the drug immediately into the urine before its return to the systemic circulation via the renal veins; thus, systemic exposure is limited.

TRT may be provided via a series of bifurcated renal artery infusion catheter and sheath systems as previously disclosed in, for example, U.S. Pat. Nos. 6,994,700 and 7,104,981 and US Pre-Grant Publications 2005/0267010 and 2006/0036218, all commonly owned and incorporated herein by reference. These percutaneous catheter systems provide for rapid and facile access to both renal arteries of a patient, in many cases simultaneously or substantially simultaneously, thus allowing for the delivery of TRT.

TRT can be applied to patients with various agents in many clinical settings to mitigate acute kidney injury. For example, TRT can be applied with fenoldopam mesylate, a dopamine DI-like agonist vasodilator (CORLOPAM® brand of Fenoldopam Mesylate Injection). Such treatment can increase glomerular filtration rate (GFR) significantly above baseline (approximately 25%) and significantly more than IV administration, with no significant effect on blood pressure, in patients with moderate to severe underlying renal dysfunction. The increase in GFR can be seen in the setting of an insult to the kidneys in the form of a nephrotoxic radio-contrast agent. In similar patients receiving the same contrast agent insult who do not receive TRT, a substantial decline in GFR can be seen (approximately 14%).

Studies in the literature have demonstrated potential positive effects of IV fenoldopam in the setting of AKI. For example, Morelli et al demonstrated improved outcomes in preventing kidney failure in septic patients. However, this study required the infusion of low-dose fenoldopam (0.09 mcg/kg/min, to prevent hypotension) over a protracted course (average of one week or more of drug infusion in the ICU) (Morelli, A. et al., Crit Care Med 33:2451-6 (2005)).

TRT with fenoldopam mesylate or dopamine can provide an effective treatment option for pre-renal AKI patients. Embodiments of the present invention encompass the use of TRT to treat AKI and facilitate dialysis removal (Allie, D. et al., J. Invasive Cardiol. 19(2):E27-30 (200)). TRT can be used with fenoldopam or dopamine, as well as other agents that have favorable renal function and renal first-pass effects. These other agents include, for example, members of the prostaglandin family, antioxidant and pH-modifying agents, free radical scavengers, recombinant human peptides/proteins such as natriuretic peptides and certain cytokines, calcium channel blockers, phosphodiesterase inhibitors, CO-releasing compounds; and prostaglandin 12 receptor.

TRT with Deferoxamine can provide an effective treatment for kidney injury due to the release of reactive oxygen species associated with reperfusion and the inflammatory cascade. Deferoxamine, a free iron chelator, is able to counter free radicals by limiting the iron, and thus preventing the amount of superoxide and hydroxyl radicals released. Intravenous infusion of deferoxamine and various other iron chelators has been associated with intense systemic hypotension. As discussed previously, hypotension is a major cause for pre-renal AKI, and thus the ability to avoid hypotension in treatment of the acute kidney injury is desirable. Also, the pharmacokinetic characteristics of deferoxamine are favorable, in that it easily passes through the kidney into the urine (leaving a reddish color). Thus, the infusion of deferoxamine via TRT is an attractive option for the treatment and prevention of reactive oxygen species associated with acute kidney injury.

Embodiments of the present invention encompass means for determining the relative kidney health of a patient. These include blood lab measurements of various substances such as creatinine (a by-product of muscle cell activity), neutrophil gelatinase associated lipocalin (NGAL, an early marker of kidney response to ischemic injury), kidney injury molecules-1 (KIM-I, an early marker of kidney response to ischemic/toxic kidney injury from cardiac surgery), N-Acetyl-f3-D-Glucosaminidase (an early marker of proximal tubular injury), Cystatin C (an early marker of decreased glomerular filtration rate), and Interleukin-18 (IL-18, an early predictive biomarker of acute kidney injury after cardiopulmonary bypass). Baseline values for patients can establish a risk of developing AKI, and attainment of threshold values or comparisons of sequential measurements at various time points after a potential renal injury can demonstrate the likelihood and extent of any renal injury that may have occurred. Thus, there are means to determine that appropriate time for intervention with TRT in order to prevent or treat AKI in certain patients. This is aided by the knowledge of the timing of the renal insult and disease progression in the case of hospital-acquired AKI.

Embodiments of the present invention encompass catheter systems and methods such as those previously described in U.S. Pat. Nos. 6,994,700 and 7,104,981 and US Pre-Grant Publications 2005/0267010 and 2006/0036218 (as previously noted and incorporated herein). These devices and methods incorporate bifurcated renal artery catheters designed and constructed so as to enable the physician user to easily and quickly access both renal arteries simultaneously with a single catheter device, often without the need for ancillary guide wires or catheters, and through a single vascular access site. This bilateral renal artery access then provides a drug delivery pathway directly to both of the patient's kidneys, allowing for administration of TRT as previously described.

The bilateral devices may be used or modified for use in the ICU setting, where many patients susceptible to or suffering from AKI or impending AKI might reside in the hospital. Relatedly, embodiments of the present invention encompass features or adaptations such as the ability to be guided into position without fluoroscopy, a heightened ability to remain stable in the body for longer periods of TRT without direct physician monitoring, and the like. Some of these features (e.g., means for renal positional stability) are described in particular in previously-disclosed co-owned US Pre-Grant Publication 2005/0267010.

In some embodiments, a method aspect revolves around specific drug treatment as delivered via TRT for the specific application of treating patients with impending or already developed AKI. Depending on the patient's condition, AKI etiology, and stage of disease (per, for example, the previously-discussed RIFLE criteria), drug treatment may be tailored to specifically address the patient's condition and provide for an effective or optimized treatment in terms of safety and efficacy.

For example, in the case of pre-renal injury due to a reduction in renal blood flow, which may be caused by systemic hypotension, cardiorenal syndrome, sepsis, hepatorenal syndrome, and the like, it is possible to administer with TRT agents such as renal vasodilators, anti-inflammatory and anti-apoptotic drugs, antioxidants, and neurohormonal regulatory agents to improve blood flow, reduce secondary injury due to reactive oxygen species (ROS) and chemotoxic injury, prevent cell apoptosis, and provide for suppression of hormones that may be causing an imbalance in the kidneys' natriuresis and diuresis. Local administration of these agents via TRT can prevent or ameliorate the systemic hypotension that often results with IV delivery, which in turn would likely exacerbate the renal blood flow problems.

As a further example, in the case of a post-renal kidney injury as may be secondary to renal vein thrombosis, TRT with anticoagulation agents can be administered, for example in conjunction with antioxidants to preclude or inhibit ROS injury as noted above. By providing the anticoagulation locally, systemic or remote hemorrhage becomes less likely.

As yet another example, in the case of sepsis, anti-inflammatory agents and antibiotics can be administered via TRT along with renal vasodilators to allow the kidney to rid the bloodstream of infectious wastes without itself becoming unduly compromised. Again, local administration of the vasodilator prevents or inhibits systemic hypotension that could worsen the clinical situation as it relates to renal function, while local anti-inflammatory delivery can allow preservation of kidney function without compromising the rest of the body's natural ability to fight the underlying infection. Local antibiotics can be used in conjunction with systemic doses in the setting of sepsis.

As yet another example, in cases of direct renal toxicity from non-renal causes, such as the systemic or remote administration of radiocontrast agents for vascular or other imaging or chemotherapy agents for various cancer conditions, where the kidney suffers injury from the toxic agents as it attempts to clear them from the circulation, TRT with vasodilators, anti-inflammatory drugs, anti-apoptotic drugs, and antioxidants can provide benefit by allowing the kidney to filter the material more quickly (due to increases in renal blood flow and GFR) and by preventing or inhibiting secondary ROS injury, while also preventing the inflammatory causes of further injury and the cell destruction due to already contracted injury.

As yet another example, in the case of renal dysfunction caused by renal cell carcinoma (RCC), patients is often treated with repeated bolus IV infusions of interleukin-2 to stimulate immune response that can attack tumor cells. However, the administration of therapeutically advantageous doses of interleukin-2 has been associated with major adverse events, most notably hypotension. The pharmacokinetic characteristics of interleukin-2 is favorable for TRT, in that it is metabolized and cleared within the kidney by both glomerular filtration and peri-tubular extraction (PROLEUKIN® Aldesleukin for Injection Package Insert & Prescribing Information Chiron Corporation (200)). As discussed previously, hypotension is a major cause for pre-renal AKI, and thus the ability to avoid hypotension in treatment of the underlying disease (in this case RCC) is desirable. Thus, interleukin-2 delivered via TRT is an attractive treatment option for patients suffering from RCC.

As yet another example, in the case of renal dysfunction caused by acute interstitial nephritis or acute glomerulonephritis, patients are often treated with repeated bolus IV infusions of methylprednisolone, hydrocortisone, or azathioprine sodium to decrease the inflammation in the kidney associated with these diseases. However, the administration of therapeutically advantageous doses of these anti-inflammatory immunosuppressant drugs has been associated with considerable systemic immune suppression and thus infections and bone marrow suppression. During the rapid onset of inflammation associated with acute glomerulonephritis and acute interstitial nephritis, the kidney requires high dosed, quick, and localized response to the inflammation. Thus, methylprednisolone, hydrocortisone, and azathioprine delivered via TRT are attractive treatment options for patients suffering from RCC.

Likewise, the TRT treatment may be determined according to the stage or severity of the AKI progression. As an example, in early stages of kidney injury, when the decline in renal function is due primarily to acute reductions in renal blood flow, TRT treatment with vasodilators and/or antioxidants can provide relief from dysfunction as this can correct or improve the blood flow imbalance and prevent or inhibit further ROS-induced cellular injury. However, should kidney injury persist prior to treatment being available, additional agents such as anti-inflammatory agents and anti-apoptotic agents may be employed to counteract injury response mechanisms within the kidney that may further exacerbate the decline in renal function. As well, neurohormonal agents can provide benefit in later-stage progression as the rest of the body begins to respond to the kidneys' decline.

Embodiments of the present invention relate to the clinical treatment of individuals susceptible to or suffering from acute kidney injury due to any number of causes. Embodiments encompass systems, devices, and means for providing drug, biologic, or other therapy or treatment comprising fluid agent delivery directly to the kidneys via their arterial blood supply. In some cases, embodiments of the invention provide a bifurcated renal artery infusion catheter device and method for its use in treating kidney injury in patients with locally-delivered drugs, biologics, and other agents. In some cases, embodiments of the present invention provide specific drug, biologic, and other agent treatment regimes for specific etiologies, severities, and degrees of progression of kidney injury. For example, a treatment method can include delivering a therapeutic agent from a therapeutic agent source through a bifurcated renal artery infusion catheter and into the first and second renal arteries, where the therapeutic agent is selected based on the stage or severity of the acute kidney injury, the cause of the acute kidney injury, or both.

Embodiments of the present invention encompass treatment for patients in at least the first three categories of AKI-Risk, Injury, and Failure by the RIFLE criteria, or Stage I, II, and III, by the AKIN criteria. Advantageously, such treatments can be provided to patients developing AKI in-hospital, where in most cases the insult that led to the AKI is known (e.g., surgical procedure, toxic insult), and thus the time course of the condition is known. Treatment can be provided to patients where permanent damage has not yet occurred to the point that kidney function cannot be at least partially recovered. Often, such patients respond where treatment is enacted in a timely manner. TRT can be used to treat or prevent acute kidney injury, particularly where patients are at risk of developing or are suffering from kidney injury related to sepsis, post-operative, renal cancers, and the like. TRT can also be used to improve symptoms and reduce complications associated with dialysis. Embodiments also encompass methods of monitoring patients prior to and in response to protocols for treating or preventing acute kidney injury. Similarly, embodiments encompass methods for decreasing symptoms or complications associated with acute kidney injury, and for improving indicators associated with kidney health. Embodiments also include methods for determining when to administer preventative or ameliorative treatments for acute kidney injury, as well as methods for evaluating and establishing regimens or protocols for preventing or inhibiting acute kidney injury.

In one aspect, embodiments of the present invention encompass methods for treating a patient suffering from acute kidney injury. An exemplary method may include placing a bifurcated renal artery infusion catheter within the abdominal aorta of the patient. The bifurcated infusion catheter can include a first renal delivery member with a first port and a second renal delivery member with a second port. The method may also include placing the first renal delivery member within a first renal artery of the patient, placing the second renal delivery member within a second renal artery of the patient, and delivering an amount of a therapeutic agent from a therapeutic agent source through the bifurcated renal artery infusion catheter and into the first and second renal arteries via the first and second ports, respectively. The acute kidney injury can be characterized by one or more clinical criteria or conditions. For example, the acute kidney injury can be characterized by an increase in serum creatinine by at least 50% over baseline, an absolute increase in serum creatinine of at least 0.3 mg/dL over baseline, a reduction in glomerular filtration rate of at least 25% compared to baseline, a decrease in urine output to 0.5 ml per kilogram of body weight or less per hour persisting for at least 6 hours, or any combination thereof. An acute kidney injury may be caused by or associated with exposure to a toxic agent such as a radiocontrast media, a non-steroidal anti-inflammatory drug (NSAID), or a chemotherapy agent. An acute kidney injury may also involve a pre renal kidney injury caused by or associated with a reduced cardiac output leading to reduced overall blood flow to the kidneys, trauma, reduced blood oxygenation, systemic toxicity caused by reaction to injury in another organ, systemic hypotension resulting from cardiorenal syndrome or acute decompensated heart failure, a reduction in circulating volume due to hemorrhage, a surgical procedure, or a reduction in local renal blood flow resulting from hepatorenal syndrome. When the acute kidney injury can be characterized according to such criteria, or when the acute kidney injury is caused or associated with by such conditions, the method may involve administration of a therapeutic agent that includes a vasodilator, an antioxidant, or both. The vasodilator can be fenoldopam mesylate or an analog or derivative thereof, dopamine or an analog or derivative thereof, or a prostaglandin or analog or derivative thereof, for example. The antioxidant can be ascorbic acid, sodium bicarbonate, or acetylcysteine, for example.

In a related aspect, an acute kidney injury can be characterized by an increase in serum creatinine by at least 100% over baseline, a reduction in glomerular filtration rate of at least 50% compared to baseline, a decrease in urine output to 0.5 ml per kilogram of body weight or less per hour persisting for at least 12 hours, or any combination thereof. When the acute kidney injury can be characterized according to such criteria, or when the acute kidney injury is caused by etiological conditions described herein, the method may involve administration of a therapeutic agent that includes a vasodilator, an antioxidant, an anti inflammatory agent, an antibiotic agent, or any combination thereof. The vasodilator can be fenoldopam mesylate or an analog or derivative thereof, dopamine or an analog or derivative thereof, or a prostaglandin or analog or derivative thereof, for example. The antioxidant can be ascorbic acid, sodium bicarbonate, or acetylcysteine, for example. The anti inflammatory agent can be a P 38 kinase inhibitor, for example. The antibiotic can be a bactericidal agent and a bacteriostatic agent, for example.

In a related aspect, an acute kidney injury can be characterized by an increase in serum creatinine by at least 200% over baseline, an absolute serum creatinine rise of at least 0.5 mg/dL to a value of at least 4 mg/dL, a reduction in glomerular filtration rate of at least 75% compared to baseline, a decrease in urine output to 0.3 ml per kilogram of body weight or less per hour persisting for at least 24 hours, a decrease in total urine output over 12 hours to 200 ml or less, or any combination thereof. When the acute kidney injury can be characterized according to such criteria, or when the acute kidney injury is caused by etiological conditions described herein, the method may involve administration of a therapeutic agent that includes a vasodilator, an antioxidant, a diuretic, an anti inflammatory agent, an antibiotic, a neurohormonally active agent, or any combination thereof. The vasodilator can be fenoldopam mesylate or an analog or derivative thereof, dopamine or an analog or derivative thereof, or a prostaglandin or analog or derivative thereof, for example. The antioxidant can be ascorbic acid, sodium bicarbonate, or acetylcysteine, for example. The diuretic can be hydrochlorothiazide (HCTZ), spironolactone, or a loop diuretic, for example. The antibiotic can be a bactericidal agent or a bacteriostatic agent, for example. The neurohormonally active agent can be a natriuretic peptide or an analog or derivative thereof, for example. In some cases, the loop diuretic may be furosemide. In some cases, the natriuretic peptide may be A type natriuretic peptide, B-type natriuretic peptide, C-type natriuretic peptide, a synthetic natriuretic peptide, or a bio-engineered natriuretic peptide.

In another aspect, an acute kidney injury can be caused at least in part by a kidney tumor or cancer. The tumor or cancer may include a renal cell carcinoma. When the acute kidney injury is caused by such conditions, the method may involve administration of a therapeutic agent that includes a vasodilator, an antioxidant, an anti inflammatory agent, a cytokine, a neurohormonally-active agent, or any combination thereof. The vasodilator can be fenoldopam mesylate or an analog or derivative thereof, dopamine or an analog or derivative thereof, or a prostaglandin or analog or derivative thereof, for example. The antioxidant can be ascorbic acid, sodium bicarbonate, or acetylcysteine, for example. The cytokine can be a lymphokine, for example. The neurohormonally active agent can be a natriuretic peptide or an analog or derivative thereof, for example. In some cases, the lymphokine can be interleukin-2 or a genetically engineered or modified version thereof, for example. In some cases, the natriuretic peptide can be A-type natriuretic peptide, B-type natriuretic peptide, C-type natriuretic peptide, a synthetic natriuretic peptide, or a bio engineered natriuretic peptide, for example.

In a further aspect, embodiments of the present invention encompass systems for treating a patient suffering from acute kidney injury. An exemplary system may include a bifurcated renal artery infusion catheter, a therapeutic agent source located externally to the patient, and a mechanism such as an infusion pump that delivers the therapeutic agent through the bifurcated renal artery catheter and directly into the right and left renal arteries of the patient. The bifurcated renal infusion catheter can be placed via a percutaneous means, and can include a distal bifurcated portion and a non-bifurcated proximal tubular portion. The infusion catheter can also include a catheter inner lumen traversing a length of the non-bifurcated proximal tubular portion, a first renal delivery member having a first distal port that is adapted to be delivered to a first delivery position within a first renal artery via a first corresponding renal ostium located at a first location along an abdominal aorta wall of an abdominal aorta in the patient, and a second renal delivery member having a second distal port that is adapted to be delivered to a second delivery position within a second renal artery via a second corresponding renal ostium located at a second location along the abdominal aorta wall that is different than the first location. Further, the infusion catheter can include a bifurcation joining the distal bifurcated portion and non-bifurcated proximal tubular portion of the catheter that provides for fluid communication between the first and second renal delivery members and the catheter inner lumen. The infusion catheter can also include a proximal coupler assembly that is adapted to be located externally of the patient when the first and second distal ports are positioned at the first and second delivery positions, respectively. The proximal coupler assembly can be coupled with the catheter inner lumen so that material can be delivered from outside the patient's body via the proximal coupler assembly, through the catheter inner lumen, through the first and second renal delivery members, through the first and second distal ports at the first and second delivery positions, respectively, and into the first and second renal arteries, also respectively. Hence, fluid agent can be delivered via the proximal coupler to each of the first and second distal ports within first and second renal arteries.

The system can be configured to deliver a treatment protocol to the patient based on the stage or severity of acute renal injury in the patient, the cause of acute renal injury in the patient, or both. For example, the system can be configured to deliver a vasodilator, an antioxidant, or both, when the acute kidney injury is characterized by an increase in serum creatinine by at least 50% over baseline, an absolute increase in serum creatinine of at least 0.3 mg/dL over baseline, a reduction in glomerular filtration rate of at least 25% compared to baseline, a decrease in urine output to 0.5 ml per kilogram of body weight or less per hour persisting for at least 6 hours, or any combination thereof. Relatedly, the system can be configured to deliver a vasodilator, an antioxidant, an anti inflammatory agent, an antibiotic agent, or any combination thereof, when the acute kidney injury is characterized by an increase in serum creatinine by at least 100% over baseline, a reduction in glomerular filtration rate of at least 50% compared to baseline, a decrease in urine output to 0.5 ml per kilogram of body weight or less per hour persisting for at least 12 hours, or any combination thereof. Similarly, a system can be configured to deliver a vasodilator, an antioxidant, an anti inflammatory agent, an antibiotic, a neurohormonally active agent, or any combination thereof, when the acute kidney injury is characterized by an increase in serum creatinine by at least 200% over baseline, an absolute serum creatinine rise of at least 0.5 mg/dL to a value of at least 4 mg/dL, a reduction in glomerular filtration rate of at least 75% compared to baseline, a decrease in urine output to 0.3 ml per kilogram of body weight or less per hour persisting for at least 24 hours, a decrease in total urine output over 12 hours to 200 ml or less, or any combination thereof. Likewise, a system can be configured to deliver vasodilator, an antioxidant, an anti inflammatory agent, a cytokine, a neurohormonally-active agent, or any combination thereof, when the acute kidney injury is caused at least in part by a kidney tumor or cancer. The tumor or cancer may include a renal cell carcinoma.

In another method aspect, embodiments of the present invention encompass techniques for treating a patient suffering from acute kidney injury that include placing a bifurcated renal artery infusion catheter within the abdominal aorta of the patient, where the bifurcated infusion catheter has a first renal delivery member with a first port and a second renal delivery member with a second port, placing the first renal delivery member within a first renal artery of the patient and placing the second renal delivery member within a second renal artery of the patient, and delivering an amount of a therapeutic agent from a therapeutic agent source through the bifurcated renal artery infusion catheter and into the first and second renal arteries via the first and second ports, respectively. The acute kidney injury can be characterized by an absolute increase in serum creatinine of more than 0.3 mg/dl within 48 hours, an increase in serum creatinine by at least 50% over baseline within 48 hours, a decrease in urine output to 0.5 ml per kilogram of body weight or less per hour persisting for at least 6 hours, or any combination thereof.

In still a further aspect, embodiments of the present invention encompass a system and method for treating a patient suffering from acute kidney injury. The acute kidney injury can be defined as one or more of an increase in serum creatinine by at least 50% over baseline, a reduction in glomerular filtration rate of at least 25% compared to baseline, or a decrease in urine output to 0.5 ml per kilogram of body weight or less per hour persisting for at least 6 hours. The system can include a bifurcated renal artery infusion catheter placed via percutaneous means. The bifurcated catheter can include a distal bifurcated portion and a proximal tubular portion, a catheter lumen traversing the length of the non-bifurcated proximal tubular portion of the catheter's length, a first renal delivery member with a first distal port that is adapted to be delivered to a first delivery position within a first renal artery via a first corresponding renal ostium located at a first location along an abdominal aorta wall of an abdominal aorta in a patient, a second renal delivery member with a second distal port that is adapted to be delivered to a second delivery position within a second renal artery via a second corresponding renal ostium located at a second location along the abdominal aorta wall that is different than the first location, a bifurcation joining the distal bifurcated portion and proximal tubular portion of the catheter and providing for fluid communication between the two renal delivery members and the catheter's inner lumen, and a proximal coupler assembly that is adapted to be located externally of the patient when the first and second distal ports are positioned at the first and second delivery positions, respectively. The proximal coupler assembly can be coupled to the catheter's inner lumen so as to deliver material from outside the patient's body via the proximal coupler assembly, through the catheter inner lumen, through the first and second renal delivery members, and into the first and second distal ports at the first and second delivery positions, respectively, and into the first and second renal arteries, also respectively. The system can also include a therapeutic agent source located externally to the patient. The therapeutic agent can include a vasodilator, an antioxidant, or both. The system can also include a means of delivering the therapeutic agent through the renal artery catheter via its proximal coupler to each of the first and second distal ports substantially within first and second renal arteries simultaneously or substantially simultaneously. In some cases, the kidney injury is caused by a toxic agent. The toxic agent can be radiocontrast media or a chemotherapy agent, for example. In some cases, the kidney injury is pre-renal in nature. For example, a pre-renal kidney injury can be caused by reduced cardiac output, reducing overall blood flow to the kidneys. In some cases, a pre-renal kidney injury is caused by systemic hypotension. Systemic hypotension can be caused by cardiorenal syndrome or acute decompensated heart failure. In some cases, a prerenal kidney injury can be caused by a reduction in circulating volume due to hemorrhage. Similarly, the pre-renal kidney injury may have occurred post surgery. Optionally, the prerenal kidney injury may have been caused by a reduction in local renal blood flow caused by hepatorenal syndrome. In some cases, the vasodilator is fenoldopam mesylate or an analog or derivative thereof, or dopamine or an analog or derivative thereof. Optionally, the vasodilator is a prostaglandin or analog or derivative thereof. In some cases, the antioxidant is sodium bicarbonate. Similarly, the antioxidant can be acetylcysteine. An intra-renally delivered agent can be dosed according to the patient's weight. Optionally, the intra-renally delivered agent can be administered at a fixed infusion rate regardless of the patient's weight. In some cases, the means of delivering the agent through the catheter is an infusion pump.

In another aspect, embodiments of the present invention include a system and method for treating a patient suffering from acute kidney injury, where the kidney injury can be defined as one or more of an increase in serum creatinine by at least 100% over baseline, a reduction in glomerular filtration rate of at least 50% compared to baseline, or a decrease in urine output to 0.5 ml per kilogram of body weight or less per hour persisting for at least 12 hours. The system can include a bifurcated renal artery infusion catheter placed via percutaneous means. The bifurcated catheter can include a distal bifurcated portion and a proximal tubular portion, a catheter lumen traversing the length of the non-bifurcated proximal tubular portion of the catheter's length, a first renal delivery member with a first distal port that is adapted to be delivered to a first delivery position within a first renal artery via a first corresponding renal ostium located at a first location along an abdominal aorta wall of an abdominal aorta in a patient, a second renal delivery member with a second distal port that is adapted to be delivered to a second delivery position within a second renal artery via a second corresponding renal ostium located at a second location along the abdominal aorta wall that is different than the first location, a bifurcation joining the distal bifurcated portion and proximal tubular portion of the catheter and providing for fluid communication between the two renal delivery members and the catheter's inner lumen, and a proximal coupler assembly that is adapted to be located externally of the patient when the first and second distal ports are positioned at the first and second delivery positions, respectively. In some cases, the proximal coupler assembly is coupled to the catheter's inner lumen so as to deliver material from outside the patient's body via the proximal coupler assembly, through the catheter inner lumen, through the first and second renal delivery members, and into the first and second distal ports at the first and second delivery positions, respectively, and into the first and second renal arteries, also respectively. The system may also include a therapeutic agent source located externally to the patient. The therapeutic agent can include one or more of a vasodilator, an antioxidant, an anti-inflammatory agent, or an antibiotic. The system can also include a means of delivering said therapeutic agent through the renal artery catheter via its proximal coupler to each of the first and second distal ports substantially within first and second renal arteries simultaneously or substantially simultaneously. In some cases, the kidney injury is caused by a toxic agent. The toxic agent can be radiocontrast media or a chemotherapy agent, for example. In some cases, the kidney injury is pre-renal in nature. For example, a pre-renal kidney injury can be caused by reduced cardiac output, reducing overall blood flow to the kidneys. In some cases, a pre-renal kidney injury is caused by systemic hypotension. Systemic hypotension can be caused by cardiorenal syndrome or acute decompensated heart failure. In some cases, a pre-renal kidney injury can be caused by a reduction in circulating volume due to hemorrhage. Similarly, the pre-renal kidney injury may have occurred post surgery. Optionally, the pre-renal kidney injury may have been caused by a reduction in local renal blood flow caused by hepatorenal syndrome. In some cases, the vasodilator is fenoldopam mesylate or an analog or derivative thereof, or dopamine or an analog or derivative thereof. Optionally, the vasodilator is a prostaglandin or analog or derivative thereof. In some cases, the antioxidant is sodium bicarbonate. Similarly, the antioxidant can be acetylcysteine. The antibiotic agent can be bacteriocidal or bacteriostatic. An intra-renally delivered agent can be dosed according to the patient's weight. Optionally, the intra-renally delivered agent can be administered at a fixed infusion rate regardless of the patient's weight. The agent can also be delivered at a variable infusion rate independent of the patient's weight. In some cases, the means of delivering the agent through the catheter is an infusion pump.

In another aspect, embodiments of the present invention encompass a system and method for treating a patient suffering from acute kidney injury, where the kidney injury is defined as one or more of an increase in serum creatinine by at least 200% over baseline, an absolute serum creatinine rise of at least 0.5 mg/dL to a value of at least 4 mg/dL, a reduction in glomerular filtration rate of at least 75% compared to baseline, a decrease in urine output to 0.3 ml per kilogram of body weight or less per hour persisting for at least 24 hours, or a decrease in total urine output over 12 hours to 200 ml or less. The system can include a bifurcated renal artery infusion catheter placed via percutaneous means. The bifurcated catheter can include a distal bifurcated portion and a proximal tubular portion, a catheter lumen traversing the length of the non-bifurcated proximal tubular portion of the catheter's length, a first renal delivery member with a first distal port that is adapted to be delivered to a first delivery position within a first renal artery via a first corresponding renal ostium located at a first location along an abdominal aorta wall of an abdominal aorta in a patient, a second renal delivery member with a second distal port that is adapted to be delivered to a second delivery position within a second renal artery via a second corresponding renal ostium located at a second location along the abdominal aorta wall that is different than the first location, a bifurcation joining the distal bifurcated portion and proximal tubular portion of the catheter and providing for fluid communication between the two renal delivery members and the catheter's inner lumen, and a proximal coupler assembly that is adapted to be located externally of the patient when the first and second distal ports are positioned at the first and second delivery positions, respectively. The proximal coupler assembly can be coupled to the catheter's inner lumen so as to deliver material from outside the patient's body via the proximal coupler assembly, through the catheter inner lumen, through the first and second renal delivery members, and into the first and second distal ports at the first and second delivery positions, respectively, and into the first and second renal arteries, also respectively. The system may also include a therapeutic agent source located externally to the patient. The therapeutic agent can include one or more of a vasodilator, an antioxidant, an anti-inflammatory agent, a diuretic, an antibiotic, or a neurohormonally active agent. The system may also include a means of delivering the therapeutic agent through the renal artery catheter via its proximal coupler to each of the first and second distal ports substantially within first and second renal arteries simultaneously. In some cases, the kidney injury is caused by a toxic agent. The toxic agent can be radiocontrast media or a chemotherapy agent, for example. In some cases, the kidney injury is pre-renal in nature. For example, a pre-renal kidney injury can be caused by reduced cardiac output, reducing overall blood flow to the kidneys. In some cases, a pre-renal kidney injury is caused by systemic hypotension. Systemic hypotension can be caused by cardiorenal syndrome or acute decompensated heart failure. In some cases, a pre-renal kidney injury can be caused by a reduction in circulating volume due to hemorrhage. Similarly, the pre-renal kidney injury may have occurred post surgery. Optionally, the pre-renal kidney injury may have been caused by a reduction in local renal blood flow caused by hepatorenal syndrome. In some cases, the vasodilator is fenoldopam mesylate or an analog or derivative thereof, or dopamine or an analog or derivative thereof. Optionally, the vasodilator is a prostaglandin or analog or derivative thereof. In some cases, the antioxidant is sodium bicarbonate. Similarly, the antioxidant can be acetylcysteine. The diuretic can be, for example, a loop diuretic such as furosemide. The antibiotic agent can be bacteriocidal or bacteriostatic. In some cases, the neurohormonally active agent is a natriuretic peptide, or an analog or derivative thereof. The natriuretic peptide may be A-type natriuretic peptide, B-type natriuretic peptide, or C-type natriuretic peptide. Relatedly, the natriuretic peptide can be a synthetic or bio-engineered natriuretic peptide. An intra-renally delivered agent can be dosed according to the patient's weight. Optionally, the intra-renally delivered agent can be administered at a fixed infusion rate regardless of the patient's weight. The agent can also be delivered at a variable infusion rate independent of the patient's weight. In some cases, the means of delivering the agent through the catheter is an infusion pump.

In still a further aspect, embodiments of the present invention encompass a system and method for treating a patient suffering from acute kidney injury, where the kidney injury is caused by a kidney tumor or cancer. The system can include a bifurcated renal artery infusion catheter placed via percutaneous means. The bifurcated catheter can include a distal bifurcated portion and a proximal tubular portion, a catheter lumen traversing the length of the non-bifurcated proximal tubular portion of the catheter's length, a first renal delivery member with a first distal port that is adapted to be delivered to a first delivery position within a first renal artery via a first corresponding renal ostium located at a first location along an abdominal aorta wall of an abdominal aorta in a patient, a second renal delivery member with a second distal port that is adapted to be delivered to a second delivery position within a second renal artery via a second corresponding renal ostium located at a second location along the abdominal aorta wall that is different than the first location, a bifurcation joining the distal bifurcated portion and proximal tubular portion of the catheter and providing for fluid communication between the two renal delivery members and the catheter's inner lumen, and a proximal coupler assembly that is adapted to be located externally of the patient when the first and second distal ports are positioned at the first and second delivery positions, respectively. The proximal coupler assembly can be coupled to the catheter's inner lumen so as to deliver material from outside the patient's body via the proximal coupler assembly, through the catheter inner lumen, through the first and second renal delivery members, and into the first and second distal ports at the first and second delivery positions, respectively, and into the first and second renal arteries, also respectively. The system may also include a therapeutic agent source located externally to the patient. The therapeutic agent can include one or more of a vasodilator, an antioxidant, an anti-inflammatory agent, a cytokine, or a neurohormonally-active agent. Further, the system may include a means of delivering said therapeutic agent through said renal artery catheter via its proximal coupler to each of the first and second distal ports substantially within first and second renal arteries simultaneously or substantially simultaneously. The kidney tumor or cancer may be related to renal cell carcinoma. The vasodilator can be fenoldopam mesylate or an analog or derivative thereof, or dopamine or an analog or derivative thereof. In some cases, the vasodilator is a prostaglandin or analog or derivative thereof. The antioxidant can be, for example, sodium bicarbonate or acetylcysteine. In some cases, the cytokine can be a lymphokine, such as interleukin-2, or a genetically engineered or modified version thereof. In some cases, the neurohormonally active agent is a natriuretic peptide, or an analog or derivative thereof. The natriuretic peptide can be A-type natriuretic peptide, B-type natriuretic peptide, or C-type natriuretic peptide. The natriuretic peptide can be a synthetic or bio-engineered natriuretic peptide. An intra-renally delivered agent can be dosed according to the patient's weight. Optionally, the intra-renally delivered agent can be administered at a fixed infusion rate regardless of the patient's weight. The agent can also be delivered at a variable infusion rate independent of the patient's weight. In some cases, the means of delivering the agent through the catheter is an infusion pump.

In another aspect, embodiments of the present invention encompass a method for treating a patient suffering from acute kidney injury, where the kidney injury is defined as one or more of an increase in serum creatinine by at least 50% over baseline, a reduction in glomerular filtration rate of at least 25% compared to baseline, or a decrease in urine output to 0.5 ml per kilogram of body weight or less per hour persisting for at least 6 hours. The method can include providing a percutaneous bifurcated renal artery infusion catheter placed within first and second renal arteries of the patient, where renal artery catheter includes a proximal coupler located externally to the patient in fluid communication with first and second delivery ports. The method can also include providing a therapeutic agent source located externally to the patient, in fluid communication with the proximal coupler of the bifurcated renal artery infusion catheter, where the therapeutic agent includes one or more of a vasodilator or an antioxidant. Further, the method can include delivering the therapeutic agent through the renal artery catheter via its proximal coupler to each of the first and second distal ports substantially within first and second renal arteries simultaneously or substantially simultaneously. In some cases, the kidney injury is caused by a toxic agent. The toxic agent can be radiocontrast media or a chemotherapy agent, for example. In some cases, the kidney injury is pre-renal in nature. For example, a pre-renal kidney injury can be caused by reduced cardiac output, reducing overall blood flow to the kidneys. In some cases, a pre-renal kidney injury is caused by systemic hypotension. Systemic hypotension can be caused by cardiorenal syndrome or acute decompensated heart failure. In some cases, a pre-renal kidney injury can be caused by a reduction in circulating volume due to hemorrhage. Similarly, the pre-renal kidney injury may have occurred post surgery. Optionally, the prerenal kidney injury may have been caused by a reduction in local renal blood flow caused by hepatorenal syndrome. In some cases, the vasodilator is fenoldopam mesylate or an analog or derivative thereof, or dopamine or an analog or derivative thereof. Optionally, the vasodilator is a prostaglandin or analog or derivative thereof. In some cases, the antioxidant is sodium bicarbonate or acetylcysteine. An intra-renally delivered agent can be dosed according to the patient's weight. Optionally, the intra-renally delivered agent can be administered at a fixed infusion rate regardless of the patient's weight. The agent can also be delivered at a variable infusion rate independent of the patient's weight. In some cases, the means of delivering the agent through the catheter is an infusion pump.

In another aspect, embodiments of the present invention provide a method for treating a patient suffering from acute kidney injury, where the kidney injury is defined as one or more of an increase in serum creatinine by at least 100% over baseline, a reduction in glomerular filtration rate of at least 50% compared to baseline, or a decrease in urine output to 0.5 ml per kilogram of body weight or less per hour persisting for at least 12 hours. The method can include providing a percutaneous bifurcated renal artery infusion catheter placed within first and second renal arteries of the patient, where the renal artery catheter includes a proximal coupler located externally to the patient in fluid communication with first and second delivery ports. The method can also include providing a therapeutic agent source located externally to the patient, in fluid communication with the proximal coupler of the bifurcated renal artery infusion catheter, where the therapeutic agent includes one or more of a vasodilator, an antioxidant, an anti-inflammatory agent, or an antibiotic. Further, the method may include delivering the therapeutic agent through the renal artery catheter via its proximal coupler to each of the first and second distal ports substantially within first and second renal arteries simultaneously. In some cases, the kidney injury is caused by a toxic agent. The toxic agent can be radiocontrast media or a chemotherapy agent, for example. In some cases, the kidney injury is pre-renal in nature. For example, a pre-renal kidney injury can be caused by reduced cardiac output, reducing overall blood flow to the kidneys. In some cases, a pre-renal kidney injury is caused by systemic hypotension. Systemic hypotension can be caused by cardiorenal syndrome or acute decompensated heart failure. In some cases, a pre-renal kidney injury can be caused by a reduction in circulating volume due to hemorrhage. Similarly, the pre-renal kidney injury may have occurred post surgery. Optionally, the prerenal kidney injury may have been caused by a reduction in local renal blood flow caused by hepatorenal syndrome. In some cases, the vasodilator is fenoldopam mesylate or an analog or derivative thereof, or dopamine or an analog or derivative thereof. Optionally, the vasodilator is a prostaglandin or analog or derivative thereof. In some cases, the antioxidant is sodium bicarbonate. Similarly, the antioxidant can be acetylcysteine. The antibiotic agent can be bacteriocidal or bacteriostatic. An intra-renally delivered agent can be dosed according to the patient's weight. Optionally, the intra-renally delivered agent can be administered at a fixed infusion rate regardless of the patient's weight. The agent can also be delivered at a variable infusion rate independent of the patient's weight. In some cases, the means of delivering the agent through the catheter is an infusion pump.

In another aspect, embodiments of the present invention encompass a method for treating a patient suffering from acute kidney injury, where the kidney injury is defined as one or more of an increase in serum creatinine by at least 200% over baseline, an absolute serum creatinine rise of at least 0.5 mg/dL to a value of at least 4 mg/dL, a reduction in glomerular filtration rate of at least 75% compared to baseline, a decrease in urine output to 0.3 ml per kilogram of body weight or less per hour persisting for at least 24 hours, or a decrease in total urine output over 12 hours to 200 ml or less. The method can include providing a percutaneous bifurcated renal artery infusion catheter placed within first and second renal arteries of the patient, where the renal artery catheter includes a proximal coupler located externally to the patient in fluid communication with first and second delivery ports. The method can also include providing a therapeutic agent source located externally to the patient, in fluid communication with the proximal coupler of the bifurcated renal artery infusion catheter, where the therapeutic agent includes one or more of a vasodilator, an antioxidant, an anti-inflammatory agent, an antibiotic, or a neurohormonally active agent. Further, the method can include delivering the therapeutic agent through the renal artery catheter via its proximal coupler to each of the first and second distal ports substantially within first and second renal arteries simultaneously or substantially simultaneously. In some cases, the kidney injury is caused by a toxic agent. The toxic agent can be radiocontrast media or a chemotherapy agent, for example. In some cases, the kidney injury is pre-renal in nature. For example, a pre-renal kidney injury can be caused by reduced cardiac output, reducing overall blood flow to the kidneys. In some cases, a pre-renal kidney injury is caused by systemic hypotension. Systemic hypotension can be caused by cardiorenal syndrome or acute decompensated heart failure. In some cases, a pre-renal kidney injury can be caused by a reduction in circulating volume due to hemorrhage. Similarly, the pre-renal kidney injury may have occurred post surgery. Optionally, the prerenal kidney injury may have been caused by a reduction in local renal blood flow caused by hepatorenal syndrome. In some cases, the vasodilator is fenoldopam mesylate or an analog or derivative thereof, or dopamine or an analog or derivative thereof. Optionally, the vasodilator is a prostaglandin or analog or derivative thereof. In some cases, the antioxidant is sodium bicarbonate. Similarly, the antioxidant can be acetylcysteine. The diuretic can be, for example, a loop diuretic such as furosemide. The antibiotic agent can be bacteriocidal or bacteriostatic. In some cases, the neurohormonally active agent is a natriuretic peptide, or an analog or derivative thereof. The natriuretic peptide may be A-type natriuretic peptide, B-type natriuretic peptide, or C-type natriuretic peptide. Relatedly, the natriuretic peptide can be a synthetic or bio-engineered natriuretic peptide. An intra-renally delivered agent can be dosed according to the patient's weight. Optionally, the intra-renally delivered agent can be administered at a fixed infusion rate regardless of the patient's weight. The agent can also be delivered at a variable infusion rate independent of the patient's weight. In some cases, the means of delivering the agent through the catheter is an infusion pump.

In still a further aspect, embodiments of the present invention encompass a method for treating a patient suffering from acute kidney injury, where the kidney injury is caused by a kidney tumor or cancer. The method can include providing a percutaneous bifurcated renal artery infusion catheter placed within first and second renal arteries of the patient, where the renal artery catheter includes a proximal coupler located externally to the patient in fluid communication with first and second delivery ports. The method can also include providing a therapeutic agent source located externally to the patient, in fluid communication with the proximal coupler of the bifurcated renal artery infusion catheter, where the therapeutic agent includes one or more of a vasodilator, an antioxidant, an anti-inflammatory agent, a cytokine, or a neurohormonally-active agent. The method may also include delivering the therapeutic agent through the renal artery catheter via its proximal coupler to each of the first and second distal ports substantially within first and second renal arteries simultaneously or substantially simultaneously. The kidney tumor or cancer may be related to renal cell carcinoma. The vasodilator can be fenoldopam mesylate or an analog or derivative thereof, or dopamine or an analog or derivative thereof. In some cases, the vasodilator is a prostaglandin or analog or derivative thereof. The antioxidant can be, for example, sodium bicarbonate or acetylcysteine. In some cases, the cytokine can be a lymphokine, such as interleukin-2, or a genetically engineered or modified version thereof. In some cases, the neurohormonally active agent is a natriuretic peptide, or an analog or derivative thereof. The natriuretic peptide can be A-type natriuretic peptide, B-type natriuretic peptide, or C-type natriuretic peptide. The natriuretic peptide can be a synthetic or bio-engineered natriuretic peptide. An intra-renally delivered agent can be dosed according to the patient's weight. Optionally, the intra-renally delivered agent can be administered at a fixed infusion rate regardless of the patient's weight. The agent can also be delivered at a variable infusion rate independent of the patient's weight. In some cases, the means of delivering the agent through the catheter is an infusion pump.

Some alternative embodiments of the present invention encompass techniques involve directing an active agent or fluid to a renal system in a patient. Such approaches may include positioning a renal device at a location within the patient's aorta that is adjacent to an ostium of at least one renal artery extending from the aorta, and segregating or dividing a first portion of aortic flow and a second portion of aortic flow from an aortic blood flow within the aorta with the renal device. Accordingly, these embodiments may include techniques discussed in, for example, U.S. Patent Publication No. 2004/0064091, the entire contents of which are hereby incorporated by reference. The technique can also include directing the first portion of aortic blood flow into the at least one renal artery, while allowing the second portion to flow across the location and downstream of the ostium. Further, the technique may include locally delivering a volume of fluid having a diagnostic, prophylactic or therapeutic agent to the at least one renal artery in the patient with the renal device. Optionally, the technique may involve positioning a medical device within an aorta of the patient upstream from an ostium of the at least one renal artery along the aorta of the patient, while locally delivering the volume of fluid to the at least one renal artery with the renal device.

Targeted Renal Therapy (TRT), or the direct delivery of therapeutic agents to the kidneys via the renal arteries, offers a way to increase the therapeutic windows of certain drugs that may have a beneficial renal effect in AKI. The benefits of TRT include the following:
(a) Delivery of agents directly to the kidneys via the renal arteries increases the local dose of the agent over that which could be achieved via systemic (intravenous, IV) delivery, as the dilution by the blood is significantly less, and
(b) Delivery directly to the kidneys can take advantage of renal first-pass, in which the kidney can clear a substantial portion of the drug immediately into the urine before its return to the systemic circulation via the renal veins; thus, systemic exposure is limited.

The referenced patents, patent applications, and scientific literature, including accession numbers to GenBank database sequences, referred to herein are hereby incorporated by reference in their entirety. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

While the above provides a full and complete disclosure of certain embodiments of the present invention, various modifications, alternate constructions and equivalents may be employed as desired. Therefore, the above description and illustrations should not be construed as limiting the invention, which is defined by the appended claims.

1. A method for the treatment of a renal cell carcinoma in a patient in need thereof, comprising:
placing a self-cannulating bifurcated catheter within the abdominal aorta of the patient, wherein the self-cannulating bifurcated catheter comprises a first branch extension having a first port and a second branch extension having a second port;
positioning the self-cannulating bifurcated catheter at or near a first renal artery and a second renal artery of the patient;
allowing the first branch extension of the self-cannulating bifurcated catheter to expand toward the first renal artery, such that the first port of the first branch extension enters the first renal artery;
allowing the second branch extension of the self-cannulating bifurcated catheter to expand toward the second renal artery, such that the second port of the second branch extension enters the second renal artery; and
infusing a fluid agent through at least one of the first and second ports of the first and second branch extensions, respectively, and into at least one of the first and second renal arteries of the patient, respectively,
wherein the fluid agent comprises a therapeutically effective amount of a cytokine.

2. The method according to claim 1, wherein the cytokine is interleukin-2.

3. The method according to claim 1, wherein the cytokine is interferon-α.

4. The method according to claim 1, wherein the fluid agent comprises interleukin-2 and interferon-α.

5. The method according to claim 1, further comprising surgically removing a renal cell carcinoma cell from the patient, wherein the fluid agent is administered to the patient following the surgical removal step.

6. The method according to claim 1, wherein the patient has been diagnosed with metastatic renal cell carcinoma.

7. A method for the treatment of a renal cell carcinoma in a patient in need thereof, comprising:
placing a bifurcated catheter within the abdominal aorta of the patient, wherein the bifurcated catheter comprises a first branch extension having a first port and a second branch extension having a second port;
positioning the bifurcated catheter at or near a first renal artery and a second renal artery of the patient;
allowing the first branch extension of the bifurcated catheter to expand toward the first renal artery, such that the first port of the first branch extension enters the first renal artery;
allowing the second branch extension of the bifurcated catheter to expand toward the second renal artery, such that the second port of the second branch extension enters the second renal artery; and
infusing a fluid agent through at least one of the first and second ports of the first and second branch extensions, respectively, and into at least one of the first and second renal arteries of the patient, respectively,
wherein the fluid agent comprises a therapeutically effective amount of an mTOR inhibitor.

8. The method according to claim 7, wherein the mTOR inhibitor is Temsirolimus.

9. The method according to claim 7, further comprising surgically removing a renal cell carcinoma cell from the patient, wherein the fluid agent is administered to the patient following the surgical removal step.

10. The method according to claim 7, wherein the patient has been diagnosed with metastatic renal cell carcinoma.

11. The method according to claim 7, wherein the bifurcated catheter is a self-cannulating bifurcated catheter.

12. A method for the treatment of a renal cell carcinoma in a patient in need thereof, comprising:
placing a self-expanding bifurcated catheter within the abdominal aorta of the patient, wherein the self-expanding bifurcated catheter comprises a first branch extension having a first port and a second branch extension having a second port;
positioning the self-expanding bifurcated catheter at or near a first renal artery and a second renal artery of the patient;
allowing the first branch extension of the self-expanding bifurcated catheter to expand toward the first renal artery, such that the first port of the first branch extension enters the first renal artery;
allowing the second branch extension of the self-expanding bifurcated catheter to expand toward the second renal artery, such that the second port of the second branch extension enters the second renal artery; and
infusing a fluid agent through at least one of the first and second ports of the first and second branch extensions, respectively, and into at least one of the first and second renal arteries of the patient, respectively,
wherein the fluid agent comprises a therapeutically effective amount of a mitotic kinesin inhibitor.

13. The method according to claim 12, wherein the mitotic kinesin inhibitor is Ispinesib.

14. The method according to claim 12, further comprising surgically removing a renal cell carcinoma cell from the patient, wherein the fluid agent is administered to the patient following the surgical removal step.

15. The method according to claim 12, wherein the patient has been diagnosed with metastatic renal cell carcinoma.

16. A method for the treatment of a renal cell carcinoma in a patient in need thereof, comprising:
placing a bifurcated catheter within the abdominal aorta of the patient, wherein the bifurcated catheter comprises a first branch extension having a first port and a second 5 branch extension having a second port;
positioning the bifurcated catheter at or near a first renal artery and a second renal artery of the patient;
allowing the first branch extension of the bifurcated catheter to expand toward the first renal artery, such that the first port of the first branch extension enters the first renal artery;
allowing the second branch extension of the bifurcated catheter to expand toward the second renal artery, such that the second port of the second branch extension enters the second renal artery; and
infusing a fluid agent into at least one of the first and second ports of the first and second branch extensions, respectively, and into at least one of the first and second renal arteries of the patient, respectively,
wherein the fluid agent comprises a therapeutically effective amount of a stem cell.

17. A method for the treatment of acute kidney injury in a patient in need thereof, comprising:
placing a self-expanding bifurcated catheter within the abdominal aorta of the patient, wherein the self-expanding bifurcated catheter comprises a first branch extension having a first port and a second branch extension having a second port;
positioning the self-expanding bifurcated catheter at or near a first renal artery and a second renal artery of the patient;
allowing the first branch extension of the self-expanding bifurcated catheter to expand toward the first renal artery, such that the first port of the first branch extension enters the first renal artery;
allowing the second branch extension of the self-expanding bifurcated catheter to expand toward the second renal artery, such that the second port of the second branch extension enters the second renal artery; and
infusing a fluid agent through the first and second ports of the first and second branch extensions, respectively, and into the first and second renal arteries of the patient, respectively,
wherein the fluid agent comprises a therapeutically effective amount of a stem cell.

18. The method according to claim 19, wherein the stem cell is a mesenchymal stem cell.

19. The method according to claim 19, wherein the stem cell is an embryonic stem cell.

20. The method according to claim 19, wherein the stem cell is an adult stem cell.
